SEQUENCE LISTING (14 sequences)

SEQ ID NO: 1 - DNA, 948 bp, Homo sapiens
  CDS (1)..(945)
atgggaagatgggtgaaccagtcctacacagatggcttcttcctcttgggcatcttttcccacagccagactgaccttgtcctcttctctgcagttatggtggtcttcacagtggccctctgtgggaatgtcctcctcatcttcctcatctacctggacgctggacttcacacccccatgtacttcttcctcagccagctctccctcatggacctcatgttggtctgtaacattgtgccaaagatggcagccaacttcctgtctggcaggaagtccatctcctttgtgggctgtggcatacaaattggcttttttgtctctcttgtgggatctgaggggctcttgctgggactcatggcttatgaccactacgtggccgttagccacccacttcactatcccatcctcatgaatcagagggtctgtctccagattactgggagctcctgggcctttgggataatagatggagtgattcagatggtggcagccatgggcttaccttactgtggctcaaggagcgtggatcactttttctgtgaggtacaagctttattgaagctggcctgtgcagacacttccctttttgacaccctcctctttgcttgctgtgtcttcatgcttctccttcccttctccatcatcatggcctcctatgcttgcatcctaggggctgtgctccgaatacgctctgctcaggcctggaaaaaagccctggccacctgctcctcccacctaacagctgtcaccctcttctatggggcagccatgttcatgtacctgaggcctaggcgctaccgggcccctagccatgacaaggtggcctctatcttctacacagtccttactcccatgctgaaccccctcatttacagcttgaggaatggggaggtgatgggggcactgaggaaggggctggaccgctgcaggattggcagccagcactga

SEQ ID NO: 2 - protein, 315 aa, Homo sapiens [sequence omitted]

SEQ ID NO: 3 - DNA, 22 bp, Artificial Sequence
  Description of Artificial Sequence PRIMER SEQUENCE
gcctctatcttctacacagtcc

SEQ ID NO: 4 - DNA, 20 bp, Artificial Sequence
  Description of Artificial Sequence PRIMER SEQUENCE
ccaaaacctataaaccatcc

SEQ ID NO: 5 - DNA, 59 bp, Artificial Sequence
  Description of Artificial Sequence PRIMER SEQUENCE
gcactagtaatacgactcactatagggagaccaccatgggaagatgggtgaaccagtcc

SEQ ID NO: 6 - DNA, 60 bp, Artificial Sequence
  Description of Artificial Sequence PRIMER SEQUENCE
gactggatcccccgggctttttttttttttttgcggccgctcagtgctggctgccaatcc

SEQ ID NO: 7 - protein, 9 aa, Artificial Sequence
  Description of Artificial Sequence MAP KINASE SUBSTRATE PEPTIDE
APRTPGGRR

SEQ ID NO: 8 - protein, 316 aa, Homo sapiens
MDNQSSTPGFLLLGFSEHPGLGRTLFVDVITSYLLTLVGNTLIILLSALDTKLHSPMYFFLSNLSFLDLCFTTSCVPQMLANLWGPKKTISFLDCSVQIFIFLSLGTTECILMKVMAFDRYVAVCQPLHYATIIHPRLCWQLASVAWVIGLVGSVVQTPSTLHLPFCPDRQVDDFVCEVPALIRLSCEDTSYNEIQVAVASVFILVVPLSLILVSYGAITWAVLRINSATAWRKAFGTCSSHLTVVTLFYSSVIAVYLQPKNPYAQGRGKFFGLFYAVGTPSLNPLVYTLRNKEIKRALRRLLGKERDSRESWRAA

SEQ ID NO: 9 - protein, 223 aa, Mus musculus
SQLSLMDLMLVCNIVPKMAVNFLSGRKSISFAGCGIQIGFFVSLVGSEGLLLGLMAYDRYVAISHPLHYPILMSQKVCLQIAGSSWAFGILDGIIQMVAAMSLPYCGSRYIDHFFCEVPALLKLACADTSLFDTLLFACCVFMLLLPFSIIVTSYARILGAVLRMHSAQSRKKALATCSSHLTAVSLFYGAAMFIYLRPRRYRAPSHDKVVSIFYTVLTPMLN

SEQ ID NO: 10 - DNA, 1070 bp, Homo Sapiens
gtgtcctcctgcatgtctcagagcatacgctgttgaactgaagaatttggagcacaatagagacatgtcactcaatcttataatttctcttatgttctcaggtgacagtgaacaactaagccatgggaagatgggtgaaccagtcctacacagatggcttcttcctcttgggcatcttttcccacagccagactgaccttgtcctcttctctgcagttatggtggtcttcacagtggccctctgtgggaatgtcctcctcatcttcctcatctacctggacgctggacttcacacccccatgtacttcttcctcagccagctctccctcatggacctcatgttggtctgtaacattgtgccaaagatggcagccaacttcctgtctggcaggaagtccatctcctttgtgggctgtggcatacaaattggcttttttgtctctcttgtgggatctgaggggctcttgctgggactcatggcttatgaccactacgtggccgttagccacccacttcactatcccatcctcatgaatcagagggtctgtctccagattactgggagctcctgggcctttgggataatagatggagtgattcagatggtggcagccatgggcttaccttactgtggctcaaggagcgtggatcactttttctgtgaggtacaagctttattgaagctggcctgtgcagacacttccctttttgacaccctcctctttgcttgctgtgtcttcatgcttctccttcccttctccatcatcatggcctcctatgcttgcatcctaggggctgtgctccgaatacgctctgctcaggcctggaaaaaagccctggccacctgctcctcccacctaacagctgtcaccctcttctatggggcagccatgttcatgtacctgaggcctaggcgctaccgggcccctagccatgacaaggtggcctctatcttctacacagtccttactcccatgctgaaccccctcatttacagcttgaggaatggggaggtgatgggggcactgaggaaggggctggaccgctgcaggattggcagccagcactga

SEQ ID NO: 11 - DNA, 43 bp, Artificial Sequence
  Description of Artificial Sequence Oligonucleotide Primer
tgaggatgggatagtgaagtgggtggctaacggccacgtagtg

SEQ ID NO: 12 - DNA, 50 bp, Artificial Sequence
  Description of Artificial Sequence Oligonucleotide Primer
tgacatgtctctattgtgctccaaattcttcagttcaacagcgtatgctc

SEQ ID NO: 13 - protein, 305 aa, Homo sapiens
NQSYTDGFFLLGIFSHSQTDLVLFSAVMVVFTVALCGNVLLIFLIYLDAGLHTPMYFFLSQLSLMDLMLVCNIVPKMAANFLSGRKSISFVGCGIQIGFFVSLVGSEGLLLGLMAYDHYVAVSHPLHYPILMNQRVCLQITGSSWAFGIIDGVIQMVAAMGLPYCGSRSVDHFFCEVQALLKLACADTSLFDTLLFACCVFMLLLPFSIIMASYACILGAVLRIRSAQAWKKALATCSSHLTAVTLFYGAAMFMYLRPRRYRAPSHDKVASIFYTVLTPMLNPLIYSLRNGEVMGALRKGLDRCR

SEQ ID NO: 14 - protein, 223 aa, Homo sapiens
SQLSLMDLMLVCNIVPKMAANFLSGRKSISFVGCGIQIGFFVSLVGSEGLLLGLMAYDHYVAVSHPLHYPILMNQRVCLQITGSSWAFGIIDGVIQMVAAMGLPYCGSRSVDHFFCEVQALLKLACADTSLFDTLLFACCVFMLLLPFSIIMASYACILGAVLRIRSAQAWKKALATCSSHLTAVTLFYGAAMFMYLRPRRYRAPSHDKVASIFYTVLTPMLN

(19) United States
(12) Patent Application Publication
(10) Pub. No.: US 2005/0074808 A1
(43) Pub. Date: Apr. 7, 2005

(54) G protein-coupled receptor expressed in brain

(75) Inventors: Gabriel Vogeli, Seattle, WA (US); Linda Wood, Portage, MI (US)

Correspondence Address: MARSHALL, GERSTEIN & BORUN LLP, 6300 SEARS TOWER, 233 S. WACKER DRIVE, CHICAGO, IL, 60606, US

(73) Assignee: PHARMACIA & UPJOHN COMPANY, Kalamazoo, MI (US)

(21) Appl. No.: 10/960,648

(22) Filed: Oct. 7, 2004

Related U.S. Application Data
(63) Continuation of application No. 09/634,109, filed on Aug. 8, 2000, now abandoned.
Application No. 09/634,109 is a continuation-in-part of application No. 09/377,563, filed on Aug. 19, 1999, now abandoned.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention provides a gene encoding a heretofore unknown G protein coupled receptor termed CON167; constructs and recombinant host cells incorporating the gene; the CON167 polypeptide encoded by the gene; antibodies to the polypeptide; and methods of making and using all of the foregoing.

The present application is a continuation-in-part of U.S. patent application Ser. No. 09/377,563, filed Aug. 19, 1999.

### FIELD OF THE INVENTION

The present invention relates generally to the fields of genetics and cellular and molecular biology. More particularly, the invention relates to a novel G protein seven transmembrane receptor that polynucleotide and polypeptide sequence that is localized to the brain.

### DESCRIPTION OF RELATED ART

Humans and other life forms are comprised of living cells. Among the mechanisms through which the cells of an organism communicate with each other and obtain information and stimuli from their environment is through cell membrane receptor molecules expressed on the cell surface. Many such receptors have been identified, characterized, and sometimes classified into major receptor superfamilies based on structural motifs and signal transduction features. Such families include (but are not limited to) ligand-gated ion channel receptors, voltage-dependent ion channel receptors, receptor tyrosine kinases, receptor protein tyrosine phosphatases, and G protein coupled receptors. The receptors are a first essential link for translating an extracellular signal into a cellular physiological response.

The G protein-coupled receptors (GPCR) form a vast superfamily of cell surface receptors which are characterized by an amino-terminal extracellular domain, a carboxyl-terminal intracellular domain, and a serpentine structure that passes through the cell membrane seven times. Hence, such receptors are sometimes also referred to as seven transmembrane (7TM) receptors. These seven transmembrane domains define three extracellular loops and three intracellular loops, in addition to the amino- and carboxyl terminal domains. The extracellular portions of the receptor have a role in recognizing and binding one or more extracellular binding partners (ligands), whereas the intracellular portions have a role in recognizing and communicating with downstream effector molecules.

The G protein coupled receptors bind a variety of ligands including calcium ions, hormones, chemokines, neuropeptides, and neurotransmitters, nucleotides, lipids, odorants, and even photons, and are important in the normal (and sometimes the aberrant) function of many cell types: [See generally A. D. Strosberg, Eur. J Biochem., 196: 1-10 (1991) and S. K. Bohm et al., Biochem J., 322: 1-18 (1997).] When a specific ligand binds to its corresponding receptor, the ligand stimulates the receptor to activate a specific heterotrimeric guanine-nucleotide-binding regulatory protein (G-protein) that is coupled to the intracellular portion of the receptor. The G protein in turn transmits a signal to an effector molecule within the cell, by either stimulating or inhibiting the activity of that effector molecule. These effector molecules include adenylate cyclase, phospholipases and ion channels. Adenylate cyclase and phospholipases are enzymes that are involved in the production of the second messenger molecules cAMP, inositol triphosphate and diacyglycerol. It is through this sequence of events that an extracellular ligand stimuli exerts intracellular changes through a G protein coupled receptor. Each such receptor has its own characteristic primary structure, expression pattern, ligand-binding profile, and intracellular effector system.

Because of the vital role of G protein coupled receptors in the communication between cells and their environment, such receptors are attractive targets for therapeutic intervention, and many drugs have been registered which are directed towards activating or antagonizing such receptors. For receptors having a known ligand, the identification of agonists or antagonists may be sought specifically for enhancing or inhibiting the action of the ligand. Some G protein coupled receptors have roles in disease pathogenesis (e.g., certain chemokine receptors that act as HIV co-receptors and may have a role in AIDS pathogenesis), and are attractive targets for therapeutic intervention even in the absence of knowledge of the natural ligand of the receptor. Other receptors are attractive targets for therapeutic intervention by virtue of their expression pattern in tissues or cell types that are attractive targets for therapeutic intervention. Examples of this latter category of receptors include receptors expressed in immune cells, for targeting to enhance immune responses to fight pathogens or cancer or inhibit autoimmune responses; and receptors expressed in the brain or other neurons, for targeting to treat schizophrenia, depression, bipolar disease, or other neurological disorders. This latter category of receptor is also useful as a marker for identifying and/or purifying (e.g., via fluorescence activated cell sorting) cellular subtypes that express the receptor. Unfortunately, only a limited number of G protein receptors from the central nervous system (CNS) are known. A need exists for identifying the existence and structure of such G protein coupled receptors.

### SUMMARY OF THE INVENTION

The present invention addresses one or more of the needs identified above in that it provides a purified polynucleotide encoding a heretofore unknown G protein coupled receptor termed CON167; constructs and recombinant host cells incorporating the polynucleotide; the CON167 polypeptide encoded by the gene; antibodies to the polypeptide; and methods of making and using all of the foregoing. As set forth in detail herein, CON167 is expressed in the brain, heart and skeletal muscle, liver, and other tissues, providing a therapeutic indication for CON167 binding partners to treat diseases associated with such tissues.

In one embodiment, the invention provides a purified and isolated CON167 seven transmembrane receptor polypeptide comprising the amino acid sequence set forth in SEQ ID NO: 2, or a fragment thereof comprising an epitope specific to the seven transmembrane receptor. By “epitope specific to” is meant a portion of the CON167 receptor that is recognizable by an antibody that is specific for CON167 seven transmembrane receptor, as defined in detail below. A preferred embodiment comprises a purified and isolated polypeptide comprising the complete amino acid sequence set forth in SEQ ID NO: 2.

Although SEQ ID NO: 2 provides a particular human sequence, the invention is intended to include within its scope other human allelic variants; non-human mammalian forms of CON167, and other vertebrate forms of CON167.

It will be appreciated that extracellular epitopes are particularly useful for generating and screening for antibodies and other binding compounds that bind to receptors such as CON167. Thus, in another preferred embodiment, the invention provides a purified and isolated polypeptide comprising at least one extracellular domain of CON167. By “extracellular domain” is meant the amino terminal extracellular domain or an extracellular loop that spans two transmembrane domains. A purified and isolated polypeptide comprising the N-terminal extracellular domain of CON167 is highly preferred. Also preferred is a purified and isolated polypeptide comprising a CON167 seven transmembrane receptor fragment selected from the group consisting of the N-terminal extracellular domain of CON167, transmembrane domains of CON167, extracellular loops connecting transmembrane domains of CON167, intracellular loops connecting transmembrane domains of CON167, the C-terminal cytoplasmic domain of CON167, and fusions thereof. Such fragments may be continuous portions of the native receptor. However, it will also be appreciated that knowledge of the CON167 gene and protein sequences as provided herein permits recombining of various domains that are not contiguous in the native protein.

In another embodiment, the invention provides purified and isolated polynucleotides (e.g., cDNA, genomic DNA, synthetic DNA, RNA, or combinations thereof, single or double stranded) that comprise a nucleotide sequence encoding the amino acid sequence of the polypeptides of the invention. Another embodiment provides a purified and isolated polynucleotide encoding the amino acid sequence of the polypeptide of the invention fused to a heterologous tag amino acid sequence. Such polynucleotides are useful for recombinantly expressing the receptor and also for detecting expression of the receptor in cells (e.g., using Northern hybridization and in situ hybridization assays. Such polynucleotides also are useful to design antisense and other molecules for the suppression of CON167 expression in a cultured cell or animal (for therapeutic purposes or to provide a model for diseases characterized by aberrant CON167 expression). Such polynucleotides are also useful to design antisense and other molecules for the suppression of CON167 expression in a cultured cell or tissue or in an animal, for therapeutic purposes or to provide a model for diseases characterized by aberrant CON167 expression. Specifically excluded from the definition of polynucleotides of the invention are entire isolated chromosomes of native host cells. A preferred polynucleotide set forth in SEQ ED NO: 1 corresponds to a naturally occurring CON167 sequence. It will be appreciated that numerous other sequences exist that also encode CON167 of SEQ ID NO: 2, due to the well-known degeneracy of the universal genetic code.

The invention also provides a purified and isolated polynucleotide comprising a nucleotide sequence that encodes a mammalian seven transmembrane receptor, wherein the polynucleotide hybridizes to the nucleotide sequence set forth in SEQ ID NO: 1 or the non-coding strand complementary thereto, under the following hybridization conditions:
(a) hybridization for 16 hours at 42° C. in a hybridization solution comprising 50% formamide, 1% SDS, 1M NaCl, 10% Dextran sulphate; and
(b) washing 2 times for 30 minutes at 60° C. in a wash solution comprising 0.1% SSC, 1% SDS. Polynucleotides that encode a human allelic variant are highly preferred.

In a related embodiment, the invention provides vectors comprising a polynucleotide of the invention. Such vectors are useful, e.g., for amplifying the polynucleotides in host cells to create useful quantities thereof. In preferred embodiments, the vector is an expression vector wherein the polynucleotide of the invention is operatively linked to a polynucleotide comprising an expression control sequence. Such vectors are useful for recombinant production of polypeptides of the invention.

In another related embodiment, the invention provides host cells that are transformed or transfected (stably or transiently) with polynucleotides of the invention or vectors of the invention. As stated above, such host cells are useful for amplifying the polynucleotides and also for expressing the CON167 seven transmembrane receptor polypeptide or fragment thereof encoded by the polynucleotide. Such host cells are useful in assays as described herein.

In still another related embodiment, the invention provides a method for producing a seven transmembrane receptor polypeptide (or fragment thereof) comprising the steps of growing a host cell of the invention in a nutrient medium and isolating the polypeptide or variant thereof from the cell or the medium. Since CON167 is a seven transmembrane receptor, it will be appreciated that, for some applications, such as certain activity assays, the preferable isolation may involve isolation of cell membranes containing the polypeptide embedded therein, whereas for other applications a more complete isolation may be preferable.

In still another embodiment, the invention provides an antibody that is specific for the CON167 seven transmembrane receptor of the invention. Antibody specificity is described in greater detail below. However, it should be emphasized that antibodies that can be generated from polypeptides that have previously been described in the literature and that are capable of fortuitously cross-reacting with CON167 (e.g., due to the fortuitous existence of a similar epitope in both polypeptides) are considered “cross-reactive” antibodies. Such cross-reactive antibodies are not antibodies that are “specific” for CON167. The determination of whether an antibody is specific for CON167 or is cross-reactive with another known receptor is made using Western blotting assays or several other assays well known in the literature. For identifying cells that express CON167 and also for modulating CON167-ligand binding activity, antibodies that specifically bind to an extracellular epitope of the CON167 seven transmembrane receptor are preferred.

In one preferred variation, the invention provides monoclonal antibodies. Hybridomas that produce such antibodies also are intended as aspects of the invention. In yet another variation, the invention provides a humanized antibody. Humanized antibodies are useful for in vivo therapeutic indications.

In another variation, the invention provides a cell-free composition comprising polyclonal antibodies, wherein at least one of the antibodies is an antibody of the invention specific for CON167. Antisera isolated from an animal is an exemplary composition, as is a composition comprising an antibody fraction of an antisera that has been resuspended in water or in another diluent, excipient, or carrier.

In still another related embodiment, the invention provides an anti-idiotypic antibody specific for an antibody that is specific for CON167.

It is well known that antibodies contain relatively small antigen binding domains that can be isolated chemically or by recombinant techniques. Such domains are useful CON167 binding molecules themselves, and also may be reintroduced into human antibodies, or fused to toxins or other polypeptides. Thus, in still another embodiment, the invention provides a polypeptide comprising a fragment of a CON167-specific antibody, wherein the fragment and the polypeptide bind to the CON167 seven transmembrane receptor. By way of non-limiting example, the invention provides polypeptides that are single chain antibodies and CDR-grafted antibodies.

Also within the scope of the invention are compositions comprising polypeptides, polynucleotides, or antibodies of the invention that have been formulated with, e.g., a pharmaceutically acceptable carrier.

The invention also provides methods of using antibodies of the invention. For example, the invention provides a method for modulating ligand binding of a CON167 seven transmembrane receptor comprising the step of contacting the seven transmembrane receptor with an antibody specific for the seven transmembrane receptor, under conditions wherein the antibody binds the receptor.

CON167 is expressed in the brain, providing an indication that aberrant CON167 signaling activity may correlate with one or more neurological disorders. The invention also provides a method for treating a neurological disorder comprising the step of administering to a mammal in need of such treatment an amount of a antibody-like polypeptide of the invention that is sufficient to modulate ligand binding of CON167 seven transmembrane receptor in neurons of the mammal. In addition to administration of antibody-like polypeptides, administration of natural ligands for CON167 as well as modulators of CON167 activity, such as small molecules that mimic, agonize or antagonize ligand-mediated CON167 signaling, are contemplated. The expression pattern provides an indication that such molecules will have utility for treating neurological and/or psychiatric diseases, including but not limited to schizophrenia, depression, anxiety, bipolar disease, affective disorders, attention deficit hyperactivity disorder/attention deficit disorder (ADHD/ADO), epilepsy, neuritis, neurasthenia, neuropathy, neuroses, Alzheimer's disease, Parkinson's disease, migraine, senile dementia, and the like. Treatment of individuals having any of these disorders is contemplated as an aspect of the invention.

CON167 also is expressed in the liver and in skeletal and heart muscle, providing an indication that aberrant CON167 expression or signaling activity may correlate with one or more cardiovascular, muscular, or hepatic disorders. The invention provides a method for treating such a disorder comprising the step of administering to a mammal in need of such treatment an amount of antibody-like polypeptide of the invention that is sufficient to modulate ligand binding of CON167 seven transmembrane receptor that is expressed in the affected heart, muscle, or liver tissue of the mammal.

The invention also provides assays to identify compounds that bind CON167 seven transmembrane receptor. One such assay comprises the steps of: (a) contacting a composition comprising CON167 seven transmembrane receptor with a compound suspected of binding CON167; and (b) measuring binding between the compound and CON167. In one variation, the composition comprises a cell expressing CON167 on its surface. In another variation, isolated CON167 or cell membranes comprising CON167 are employed. The binding may be measured directly, e.g., using a labeled compound, or may be measured indirectly by several techniques, including measuring intracellular signaling of CON167 induced by the compound (or measuring changes in the level of CON167 signaling).

The invention also provides a method for identifying a modulator of binding between a CON167 seven transmembrane receptor and a CON167 binding partner, comprising the steps of: (a) contacting a CON167 binding partner and a composition comprising a CON167 seven transmembrane receptor in the presence and in the absence of a putative modulator compound; (b) detecting binding between the binding partner and the CON167; and (c) identifying a putative modulator compound in view of decreased or increased binding between the binding partner and the CON167 in the presence of the putative modulator, as compared to binding in the absence of the putative modulator.

CON167 binding partners that stimulate CON167 are useful as agonists in disease states characterized by insufficient CON167 signaling (e.g., as a result of insufficient expression of active CON167 ligand). CON167 binding partners that block ligand-mediated CON167 signaling are useful as CON167 antagonists to treat disease states characterized by excessive CON167 signaling.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWING

FIG. 1 depicts an alignment of a portion of the CON167 amino acid sequence and a portion of the amino acid sequence of the human olfactory receptor FAT11 (Genbank Accession No. L35475, SEQ ID NO: 8). Identical residues and residues of similar character (+) are indicated.

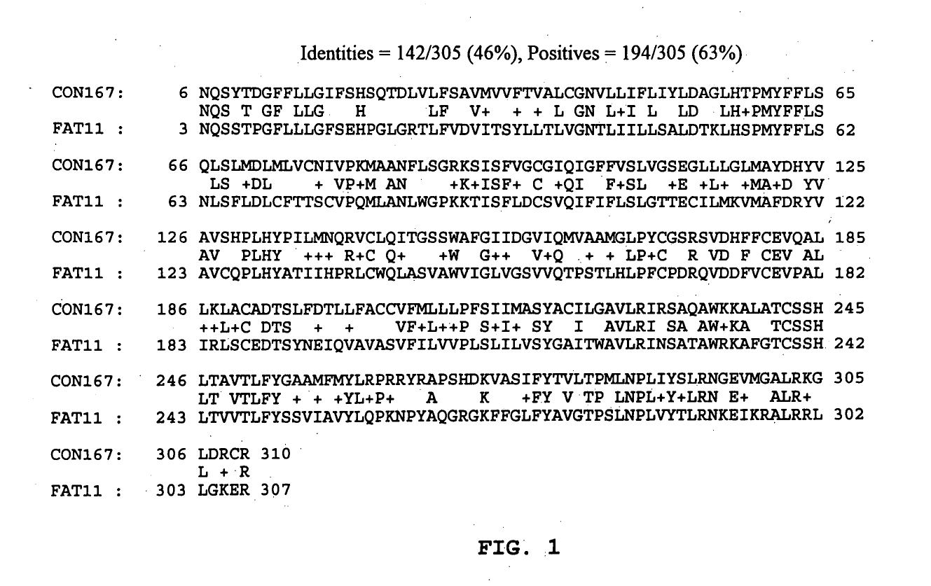

FIG. 2 depicts an alignment of a portion of the CON167 amino acid sequence with a portion of the amino acid sequence of the murine olfactory receptor G7 (Genbank AF102537, SEQ ID NO: 9). Identical residues and residues of similar character (+) are indicated.

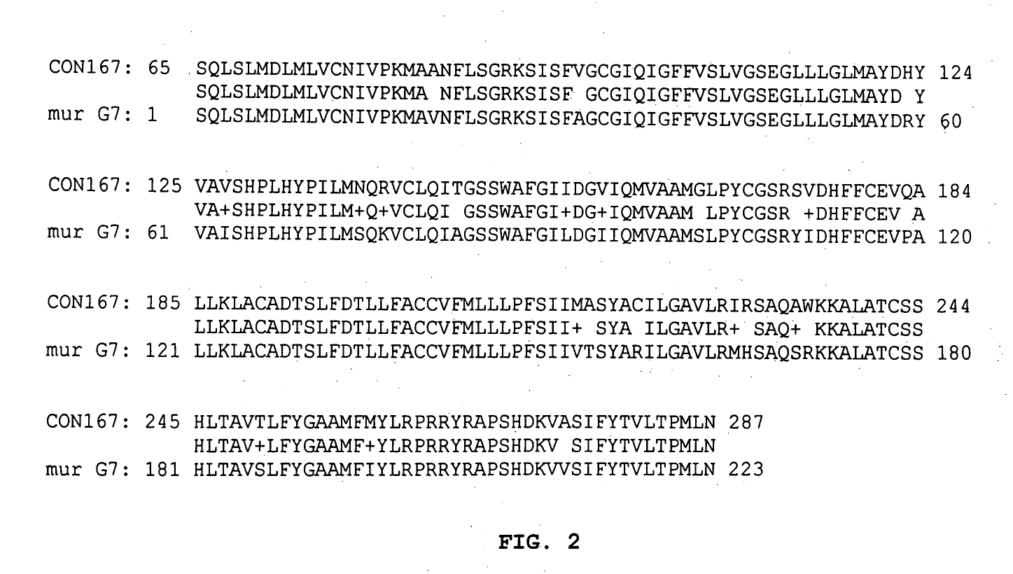

### DETAILED DESCRIPTION OF THE INVENTION

The present invention provides purified and isolated polynucleotides (e.g., DNA sequences and RNA transcripts, both sense and complementary antisense strands, both single and double stranded, including splice variants thereof) encoding a human G protein coupled receptor referred to herein as CON167. DNA polynucleotides of the invention include genomic DNA, cDNA, and DNA that has been chemically synthesized in whole or in part. “Synthesized” as used herein and understood in the art, refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means.

Genomic DNA of the invention comprises the protein coding region for a polypeptide of the invention and is also intended to include allelic variants thereof. It is widely understood that, for many genes, genomic DNA is transcribed into RNA transcripts that undergo one or more splicing events wherein intron (i.e., non-coding regions) of the transcripts are removed, or “spliced out.” RNA transcripts that can be spliced by alternative mechanisms, and therefore be subject to removal of different RNA sequences but still encode a CON167 polypeptide, are referred to in the art as splice variants which are embraced by the invention. Splice variants comprehended by the invention therefore are encoded by the same original genomic DNA sequences but arise from distinct mRNA transcripts. Allelic variants are modified forms of a wild type gene sequence, the modification resulting from recombination during chromosomal segregation or exposure to conditions which give rise to genetic mutation. Allelic variants, like wild type genes, are naturally occurring sequences (as opposed to non-naturally occurring variants which arise from in vitro manipulation).

The invention also comprehends cDNA that is obtained through reverse transcription of an RNA polynucleotide encoding CON167 (conventionally followed by second strand synthesis of a complementary strand to provide a double-stranded DNA).

A preferred DNA sequence encoding a human CON167 polypeptide is set out in SEQ ID NO: 1. The worker of skill in the art will readily appreciate that the preferred DNA of the invention comprises a double stranded molecule, for example the molecule having the sequence set forth in SEQ ID NO: 1 along with the complementary molecule (the “non-coding strand” or “complement”) having a sequence deducible from the sequence of SEQ ID NO: 1, according to Watson-Crick base pairing rules for DNA. Also preferred are other polynucleotides encoding the CON167 polypeptide of SEQ ID NO: 2, which differ in sequence from the polynucleotide of SEQ ID NO: 1 by virtue of the well-known degeneracy of the universal genetic code.

The invention further embraces species, preferably mammalian, homologs of the human CON167 DNA. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with human DNA of the invention. Percent sequence “homology” with respect to polynucleotides of the invention is defined herein as the percentage of nucleotide bases in the candidate sequence that are identical to nucleotides in the CON167 sequence set forth in SEQ ID NO: 1, after aligning the sequences and introducing gaps, if necessary, to achieve the maximum percent sequence identity.

The polynucleotide sequence information provided by the invention makes possible large scale expression of the encoded polypeptide by techniques well known and routinely practiced in the art. Polynucleotides of the invention also permit identification and isolation of polynucleotides encoding related CON167 polypeptides, such as human allelic variants and species homologs, by well known techniques including Southern and/or Northern hybridization, and polymerase chain reaction (PCR). Examples of related polynucleotides include human and non-human genomic sequences, including allelic variants, as well as polynucleotides encoding polypeptides homologous to CON167 and structurally related polypeptides sharing one or more biological, immunological, and/or physical properties of CON167. Non-human species genes encoding proteins homologous to CON167 can also be identified by Southern and/or PCR analysis and are useful in animal models for CON167 disorders. Knowledge of the sequence of a human CON167 DNA also makes possible through use of Southern hybridization or polymerase chain reaction (PCR) the identification of genomic DNA sequences encoding CON167 expression control regulatory sequences such as promoters, operators, enhancers, repressors, and the like. Polynucleotides of the invention are also useful in hybridization assays to detect the capacity of cells to express CON167. Polynucleotides of the invention may also be the basis for diagnostic methods useful for identifying a genetic alteration(s) in a CON167 locus that underlies a disease state or states, which information is useful both for diagnosis and for selection of therapeutic strategies.

The disclosure herein of a full length polynucleotide encoding a CON167 polypeptide makes readily available to the worker of ordinary skill in the art every possible fragment of the full length polynucleotide. The invention therefore provides fragments of CON167-encoding polynucleotides comprising at least 14-15, and preferably at least 18, 20, 25, 50, or 75 consecutive nucleotides of a polynucleotide encoding CON167. Preferably, fragment polynucleotides of the invention comprise sequences unique to the CON167-encoding polynucleotide sequence, and therefore hybridize under highly stringent or moderately stringent conditions only (i.e., “specifically”) to polynucleotides encoding CON167 (or fragments thereof). Fragments derived from nucleotides 1 to 769 of SEQ ID NO: 1, that are upstream of the sequence found in clone 2939091H1 are highly preferred. Polynucleotide fragments of genomic sequences of the invention comprise not only sequences unique to the coding region, but also include fragments of the full length sequence derived from introns, regulatory regions, and/or other non-translated sequences. Sequences unique to polynucleotides of the invention are recognizable through sequence comparison to other known polynucleotides, and can be identified through use of alignment programs routinely utilized in the art, e.g., those made available in public sequence databases. Such sequences also are recognizable from Southern and Northern hybridization analyses to determine the number of fragments of genomic DNA and RNA to which a polynucleotide will hybridize. Polynucleotides of the invention can be labeled in a manner that permits their detection, including radioactive, fluorescent, and enzymatic labeling.

Fragment polynucleotides are particularly useful as probes for detection of full length or other fragment CON167 polynucleotides. One or more fragment polynucleotides can be included in kits that are used to detect the presence of a polynucleotide encoding CON167, or used to detect variations in a polynucleotide sequence encoding CON167.

The invention also embraces DNAs encoding CON167 polypeptides which DNAs hybridize under moderately stringent or high stringency conditions to the non-coding strand, or complement, of the polynucleotide in SEQ ID NO: 1.

Exemplary highly stringent hybridization conditions are as follows: hybridization at 42° C. in a hybridization solution comprising 50%. formamide, 1% SDS, 1M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel, et al. (Eds.), Protocols in Molecular Biology, John Wiley & Sons (1994), pp.6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

Autonomously replicating recombinant expression constructs such as plasmid and viral DNA vectors incorporating polynucleotides of the invention are also provided. Expression constructs wherein CON167-encoding polynucleotides are operatively linked to an endogenous or exogenous expression control DNA sequence and a transcription terminator are also provided. Expression control DNA sequences include promoters, enhancers, and operators, and are generally selected based on the expression systems in which the expression construct is to be utilized. Preferred promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and preferably promote, homologous recombination in a host cell. Preferred constructs of the invention also include sequences necessary for replication in a host cell.

Expression constructs are preferably utilized for production of an encoded protein, but may also be utilized simply to amplify a CON167-encoding polynucleotide sequence.

According to another aspect of the invention, host cells are provided, including prokaryotic and eukaryotic cells, comprising a polynucleotide of the invention (or vector of the invention) in a manner which permits expression of the encoded CON167 polypeptide. Polynucleotides of the invention may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, and mammalian cells systems.

Host cells of the invention are a valuable source of immunogen for development of antibodies specifically immunoreactive with CON167. Host cells of the invention are also useful in methods for large scale production of CON167 polypeptides wherein the cells are grown in a suitable culture medium and the desired polypeptide products are isolated from the cells or from the medium in which the cells are grown by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those wherein the desired protein is expressed and purified as a fusion protein having a specific tag, label, or chelating moiety that is recognized by a specific binding partner or agent. The purified protein can be cleaved to yield the desired protein, or be left as an intact fusion protein. Cleavage of the fusion component may produce a form of the desired protein having additional amino acid residues as a result of the cleavage process.

Knowledge of CON167 DNA sequences allows for modification of cells to permit, or increase, expression of endogenous CON167. Cells can be modified (e.g., by homologous recombination) to provide increased expression by replacing, in whole or in part, the naturally occurring CON167 promoter with all or part of a heterologous promoter so that the cells express CON167 at higher levels. The heterologous promoter is inserted in such a manner that it is operatively linked to endogenous CON167 encoding sequences. [See, for example, PCT International Publication No. WO 94/12650, PCT International Publication No. WO 92/20808, and PCT International Publication No. WO 91/09955.] It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the CON167 coding sequence, amplification of the marker DNA by standard selection methods results in co-amplification of the CON167 coding sequences in the cells.

The DNA sequence information provided by the present invention also makes possible the development through, e.g. homologous recombination or “knock-out” strategies [Capecchi, Science 244:1288-1292 (1989)], of animals that fail to express functional CON167 or that express a variant of CON167. Such animals (especially small laboratory animals such as rats, rabbits, and mice) are useful as models for studying the in vivo activities of CON167 and modulators of CON167.

Also made available by the invention are anti-sense polynucleotides which recognize and hybridize to polynucleotides encoding CON167. Full length and fragment anti-sense polynucleotides are provided. Fragment anti-sense molecules of the invention include (i) those which specifically recognize and hybridize to CON167 RNA (as determined by sequence comparison of DNA encoding CON167 to DNA encoding other known molecules). Identification of sequences unique to CON167-encoding polynucleotides can be deduced through use of any publicly available sequence database, and/or through use of commercially available sequence comparison programs, and can be further verified by hybridization analysis against genomic DNA. After identification of the desired sequences, isolation through restriction digestion or amplification using any of the various polymerase chain reaction techniques well known in the art can be performed. Anti-sense polynucleotides are particularly relevant to regulating expression of CON167 by those cells expressing CON167 mRNA.

Antisense nucleic acids (preferably 10 to 20 base pair oligonucleotides) capable of specifically binding to CON167 expression control sequences or CON167 RNA are introduced into cells (e.g., by a viral vector or colloidal dispersion system such as a liposome). The antisense nucleic acid binds to the CON167 target nucleotide sequence in the cell and prevents transcription or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end. Suppression of CON167 expression at either the transcriptional or translational level is useful to general cellular and/or animal models for diseases characterized by aberrant expression. Suppression of CON167 expression at either the transcriptional or translational level is useful to generate cellular animal models for diseases characterized by aberrant CON167 expression.

The CON167 sequences taught in the present invention facilitate the design of novel transcription factors for modulating CON167 expression in native cells and animals, and cells transformed or transfected with CON167 polynucleotides. For example, the Cys₂-His₂ zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular CON167 target sequence of the present invention facilitates the engineering of zinc finger proteins specific for the target sequence using known methods such as a combination of structure-based modeling and screening of phage display libraries [Segal et al., Proc. Natl. Acad. Sc.i U.S.A., 96: 2758-2763 (1999); Liu et al., Proc. Natl. Acad. Sci. U.S.A., 94: 5525-30 (1997); Greisman and Pabo, Science, 275: 657-61 (1997); Choo et al., J. Mol. Biol., 273: 525-32 (1997)]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., Proc. Natl. Acad. Sci. U.S.A., 96: 2758-2763 (1999)]. The artificial zinc finger repeats, designed based on CON167 sequences, are fused to activation or repression domains to promote or suppress CON167 expression [Liu et al., Proc. Natl. Acad. Sci. U.S.A., 94: 5525-30 (1997)]. Alternatively, the zinc finger domains can be fused to the TATA box-binding factor (TBP) with varying lengths of linker region between the zinc finger peptide and the TBP to create either transcriptional activators or repressors [Kim et al., Proc. Natl. Acad. Sci. U.S.A., 94: 3616-3620 (1997)]. Such proteins, and polynucleotides that encode them, have utility for modulating CON167 expression in vivo in both native cells, animals and humans; and/or cells transfected with CON167-encoding sequences. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., Proc. Natl. Acad. Sci. U.S.A, 96:9521-6 (1999); Wu et al., Proc. Natl. Acad. Sci. U.S.A., 92:344-348 (1995)]. The present invention contemplates methods of designing such transcription factors based on the gene sequence of the invention, as well as customized zinc finger proteins, that are useful to modulate CON167 expression in cells (native or transformed) whose genetic complement includes these sequences.

The invention also provides purified and isolated mammalian CON167 polypeptides encoded by a polynucleotide of the invention. Presently preferred is a human CON167 polypeptide comprising the amino acid sequence set out in SEQ ID NO: 2.

The invention also embraces polypeptides that have at least 99%, at least 95%, at least 90%, at least 85%, at least 80%, at least 75%, at least 70%, at least 65%, at least 60%, at least 55% or at least 50% identity and/or homology to the preferred polypeptide of the invention. Percent amino acid sequence “identity” with respect to the preferred polypeptide of the invention is defined herein as the percentage of amino acid residues in the candidate sequence that are identical with the residues in the CON167 sequence after aligning both sequences and introducing gaps, if necessary, to achieve the maximum percent sequence identity, and not considering any conservative substitutions as part of the sequence identity. Percent sequence “homology” with respect to the preferred polypeptide of the invention is defined herein as the percentage of amino acid residues in the candidate sequence that are identical with the residues in the CON167 sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment [Dayhoff, in Atlas of Protein Sequence and Structure, Vol. 5, p. 124, National Biochemical Research Foundation, Washington, D.C. (1972), incorporated herein by reference].

Polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated forms of CON167 polypeptides are embraced.

The invention also embraces variant (or analog) CON167 polypeptides. In one example, insertion variants are provided wherein one or more amino acid residues supplement a CON167 amino acid sequence. Insertions may be located at either or both termini of the protein, or may be positioned within internal regions of the CON167 amino acid sequence. Insertional variants with additional residues at either or both termini can include for example, fusion proteins and proteins including amino acid tags or labels.

Insertion variants include CON167 polypeptides wherein one or more amino acid residues are added to a CON167 amino acid sequence, or to a biologically active fragment thereof.

Variant products of the invention also include mature CON167 products, i.e., CON167 products wherein leader or signal sequences are removed, with additional amino terminal residues. The additional amino terminal residues may be derived from another protein, or may include one or more residues that are not identifiable as being derived from a specific proteins. CON167 products with an additional methionine residue at position-1 (Met⁻¹-CON167) are contemplated, as are variants with additional methionine and lysine residues at positions-2 and -1 (Met⁻²-Lys⁻¹-CON167). Variants of CON167 with additional Met, Met-Lys, Lys residues (or one or more basic residues in general) are particularly useful for enhanced recombinant protein production in bacterial host cell.

The invention also embraces CON167 variants having additional amino acid residues which result from use of specific expression systems. For example, use of commercially available vectors that express a desired polypeptide as part of glutathione-S-transferase (GST) fusion product provides the desired polypeptide having an additional glycine residue at position-1 after cleavage of the GST component from the desired polypeptide. Variants which result from expression in other vector systems are also contemplated.

Insertional variants also include fusion proteins wherein the amino and/or carboxy termini of CON167 is fused to another polypeptide.

In another aspect, the invention provides deletion variants wherein one or more amino acid residues in a CON167 polypeptide are removed. Deletions can be effected at one or both termini of the CON167 polypeptide, or with removal of one or more residues within the CON167 amino acid sequence. Deletion variants, therefore, include all fragments of a CON167 polypeptide.

The invention also embraces polypeptide fragments of the sequence set out in SEQ ID NO: 2wherein the fragments maintain biological (e.g., ligand binding and/or intracellular signaling) immunological properties of a CON167 polypeptide. Fragments comprising at least 5, 10, 15 20, 25, 30, 35, or 40 consecutive amino acids of SEQ ID NO: 2 are comprehended by the invention. Preferred polypeptide fragments display antigenic properties unique to or specific for human CON167 and its allelic and species homologs. Fragments of the invention having the desired biological and immunological properties can be prepared by any of the methods well known and routinely practiced in the art.

In still another aspect, the invention provides substitution variants of CON167 polypeptides. Substitution variants include those polypeptides wherein one or more amino acid residues of a CON167 polypeptide are removed and replaced with alternative residues. In one aspect, the substitutions are conservative in nature, however, the invention embraces substitutions that ore also non-conservative. Conservative substitutions for this purpose may be defined as set out in: Tables A, B, or C below.

Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A (from WO 97/09433, page 10, published Mar. 13, 1997 (PCT/GB96/02197, filed Sep. 6, 1996), immediately below.

TABLE I
Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, [Biochemistry, Second Edition; Worth Publishers, Inc. NY:NY (1975), pp.71-77] as set out in Table B, immediately below.

TABLE B
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still an another alternative, exemplary conservative substitutions are set out in Table C, immediately below.

TABLE C
Conservative Substitutions III

| Original | |
|---|---|
| Residue | Exemplary Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

CON167 variants that display ligand binding properties of native CON167 and are expressed at higher levels, and variants that provide for constitutive active receptor are particularly useful in assays of the invention. Such variants also are useful in cellular and animal models for diseases characterized by aberrant CON167 expression/activity.

It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties. Such derivatives may be prepared to increase circulating half-life of a polypeptide, or may be designed to improve targeting capacity for the polypeptide to desired cells, tissues, or organs.

Similarly, the invention further embraces CON167 polypeptides that have been covalently modified to include one or more water soluble polymer attachments such as polyethylene glycol, polyoxyethylene glycol, or polypropylene glycol.

In a related embodiment, the present invention provides compositions comprising purified polypeptides of the invention. Preferred compositions comprise, in addition to the polypeptide of the invention, a pharmaceutically acceptable (i.e., sterile and non-toxic) liquid, semisolid, or solid diluents that serve as pharmaceutical vehicles, excipients, or media. Any diluent known in the art may be used. Exemplary diluents include, but are not limited to, water, saline solutions, polyoxyethylene sorbitan monolaurate, magnesium stearate, methyl- and propylhydroxybenzoate, talc, alginates, starches, lactose, sucrose, dextrose, sorbitol, mannitol, glycerol, calcium phosphate, mineral oil, and cocoa butter.

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for CON167 or fragments thereof. Preferred antibodies of the invention are human antibodies which can be produced and identified according to methods described in WO93/11236, published Jun. 20, 1993, which is incorporated herein by reference in its entirety. Antibody fragments, including Fab, Fab′, F(ab′)₂, and Fᵥ, are also provided by the invention. The term “specific for,” when used to describe antibodies of the invention, indicates that the variable regions of the antibodies of the invention recognize and bind CON167 polypeptides exclusively (i.e., able to distinguish CON167 polypeptides from other known GPCR polypeptides by virtue of measurable differences in binding affinity, despite the possible existence of localized sequence identity, homology, or similarity between CON167 and such polypeptides). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the CON167 polypeptides of the invention are also contemplated, provided that the antibodies are, first and foremost, specific for CON167 polypeptides. Antibodies of the invention can be produced using any method well known and routinely practiced in the art.

Non-human antibodies may be humanized by any methods known in the art. In one method, the non-human CDRs are inserted into a human antibody or consensus antibody framework sequence. Further changes can then be introduced into the antibody framework to modulate affinity or immunogenicity.

Antibodies of the invention are useful for, for example, therapeutic purposes (by modulating activity of CON167), diagnostic purposes to detect or quantitate CON167, as well as purification of CON167. Kits comprising an antibody of the invention for any of the purposes described herein are also comprehended. In general, a kit of the invention also includes a control antigen for which the antibody is immunospecific.

Specific binding molecules, including natural ligands and synthetic compounds, can be identified or developed using isolated or recombinant CON167 products, CON167 variants, or preferably, cells expressing such products. Binding partners are useful for purifying CON167 products and detection or quantification of CON167 products in fluid and tissue samples using known immunological procedures. Binding molecules are also manifestly useful in modulating (i.e., blocking, inhibiting or stimulating) biological activities of CON167, especially those activities involved in signal transduction.

The DNA and amino acid sequence information provided by the present invention also makes possible identification of binding partner compounds with which a CON167 polypeptide or polynucleotide will interact. Methods to identify binding partner compounds include solution assays, in vitro assays wherein CON167 polypeptides are immobilized, and cell based assays. Identification of binding partner compounds of CON167 polypeptides provides candidates for therapeutic or prophylactic intervention in pathologies associated with CON167 normal and aberrant biological activity.

The invention includes several assay systems for identifying CON167 binding partners. In solution assays, methods of the invention comprise the steps of (a) contacting a CON167 polypeptide with one or more candidate binding partner compounds and (b) identifying the compounds that bind to the CON167 polypeptide. Identification of the compounds that bind the CON167 polypeptide can be achieved by isolating the CON167 polypeptide/binding partner complex, and separating the CON167 polypeptide from the binding partner compound. An additional step of characterizing the physical, biological, and/or biochemical properties of the binding partner compound in also comprehended in another embodiment of the invention. In one aspect, the CON167 polypeptide/binding partner complex is isolated using a antibody immunospecific for either the CON167 polypeptide or the candidate binding partner compound.

In still other embodiments, either the CON167 polypeptide or the candidate binding partner compound comprises a label or tag that facilitates its isolation, and methods of the invention to identify binding partner compounds include a step of isolating the CON167 polypeptide/binding partner complex through interaction with the label or tag. An exemplary tag of this type is a poly-histidine sequence, generally around six histidine residues, that permits isolation of a compound so labeled using nickel chelation. Other labels and tags, such as the FLAG® tag (Eastman Kodak, Rochester, N.Y.), well known and routinely used in the art, are embraced by the invention.

In one variation of an in vitro assay, the invention provides a method comprising the steps of (a) contacting an immobilized CON167 polypeptide with a candidate binding partner compound and (b) detecting binding of the candidate compound to CON167 polypeptide. In an alternative embodiment, the candidate binding partner compound is immobilized and binding of CON167 is detected. Immobilization is accomplished using any of the methods well known in the art, including covalent bonding to a support, a bead, or a chromatographic resin, as well as non-covalent, high affinity interaction such as antibody binding, or use of streptavidin/biotin binding wherein the immobilized compound includes a biotin moiety. Detection of binding can be accomplished (i) using a radioactive label on the compound that is not immobilized, (ii) using of a fluorescent label on the non- immobilized compound, (iii) using an antibody immunospecific for the non-immobilized compound, (iv) using a label on the non-immobilized compound that excites a fluorescent support to which the immobilized compound is attached, as well as other techniques well known and routinely practiced in the art.

The invention also provides cell-based assays to identify binding partner compounds of a CON167 polypeptide. In one embodiment, the invention provides a method comprising the steps of contacting a CON167 polypeptide expressed on the surface of a cell with a candidate binding partner compound and detecting binding of the candidate binding partner compound to the CON167 polypeptide. In a preferred embodiment, the detection comprises detecting a calcium flux or other physiological cellular event caused by the binding of the molecule.

Agents that modulate (i.e., increase, decrease, or block) CON167 activity or expression may be identified by incubating a putative modulator with a cell expressing a CON167 polypeptide or polynucleotide and determining the effect of the putative modulator on CON167 activity or expression. The selectivity of a compound that modulates the activity of CON167 can be evaluated by comparing its effects on CON167 to its effect on other GPCR compounds. Selective modulators may include, for example, antibodies and other proteins, peptides, or organic molecules which specifically bind to a CON167 polypeptide or a CON167-encoding nucleic acid. Modulators of CON167 activity will be therapeutically useful in treatment of diseases and physiological conditions in which normal or aberrant CON167 activity is involved.

Methods of the invention to identify modulators include variations on any of the methods described above to identify binding partner compounds, the variations including techniques wherein a binding partner compound has been identified and the binding assay is carried out in the presence and absence of a candidate modulator. A modulator is identified in those instances where binding between the CON167 polypeptide and the binding partner compound changes in the presence of the candidate modulator compared to binding in the absence of the candidate modulator compound. A modulator that increases binding between the CON167 polypeptide and the binding partner compound is described as an enhancer or activator, and a modulator that decreases binding between the CON167 polypeptide and the binding partner compound is described as an inhibitor.

The invention also comprehends high throughput screening (HTS) assays to identify compounds that interact with or inhibit biological activity (i.e., inhibit enzymatic activity, binding activity, etc.) of a CON167 polypeptide. HTS assays permit screening of large numbers of compounds in an efficient manner. Cell-based HTS systems are contemplated to investigate CON167 receptor-ligand interaction. HTS assays are designed to identify “hits” or ‘lead compounds” having the desired property, from which modifications can be designed to improve the desired property. Chemical modification of the “hit” or “lead compound” is often based on an identifiable structure/activity relationship between the “hit” and the CON167 polypeptide.

Mutations in the CON167 gene that result in loss of normal function of the CON167 gene product underlie CON167-related human disease states. The invention comprehends gene therapy to restore CON167 activity to treat those disease states. Delivery of a functional CON167 gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp.25-20 (1998). For additional reviews of gene therapy technology see Friedmann, Science, 244: 1275-1281 (1989); Verma, Scientific American: 68-84-(1990); and Miller, Nature, 357: 455-460 (1992). Alternatively, it is contemplated that in other human disease states, preventing the expression of or inhibiting the activity of CON167 will be useful in treating the disease states. It is contemplated that antisense therapy or gene therapy could be applied to negatively regulate the expression of CON167.

Additional features of the invention will be apparent from the following Examples.

### EXAMPLE 1

### Cloning of CON167 G Protein Coupled Receptor

A. Database Search

The Incyte and Genbank expressed sequence tag (EST) databases were searched with the NCBI program Blastall using all known GPCR's as query sequences to find patterns suggestive of novel G protein coupled receptors. Positive hits from the find-pattern program were further analyzed with the GCG program BLAST to determine which ones were the most likely candidates to encode G-protein coupled receptors using the standard (default) alignment produced by BLAST as a guide.

This searching identified Clone 2939091H1 in the Incyte database as an interesting candidate sequence. The 2939091H1 clone was obtained and sequenced using standard techniques. From the sequence it was deduced that Clone 2939091H contained only the seventh transmembrane region (TM7) of a GPCR. Referring to SEQ ID NO: 1, the nucleotide sequence of Clone 2939091H1 corresponds to bases 770 to 948 of what was eventually determined to be the complete sequence of a novel seven transmembrane receptor. A database search with this partial sequence showed a 56% match to an olfactory receptor.

B. Screening of a Phage Library to Obtain a Full-Length GPCR Clone

Based on the complete sequence of Clone 2939091H1, two oligonucleotide primers were designed to screen a genomic library in an attempt to isolate a full-length clone of a novel GPCR:

(SEQ ID NO: 3)

Primer LW1129: 5′-GCCTCTATCTTCTACACAGTCC-3′,
and

(SEQ ID NO: 4)

Primer LW1130: 5′-CCAAAACCTATAAACCATCC-3′.

These primers were designed to amplify a 251 base pair portion of a clone containing the partial putative GPCR sequence found in Clone 29399991H1 (assuming the absence of introns in this region).

To prepare a suitable library for screening, two microliters of a human genomic library (˜10⁸ plaque forming units per milliliter (PFU/ml) (Clontech) were added to 6 ml of an overnight culture of K802 cells (ClonTech), then distributed as 250 μl aliquots into each of 24 tubes; The tubes were incubated at 37° C. for 15 minutes, and then seven millimeters of 0.8% agrarose were added to each tube. After mixing, the contents of the tubes were poured onto LB plates and incubated overnight at 37° C.

A BA85 nitrocellulose filter (Schleicher & Schuell) was placed on top of each of the confluent plates for 10-15 minutes. The filter was removed, placed phage side up in a petri dish, and covered with 4 ml of SM media (0.1 M NaCl, 8.1 μM MgSO₄.7H₂O, 50 mM Tris-Cl, pH 7.5, 0.001% gelatin) for 15 minutes to elute the phage. One milliliter of SM was removed from each plate, centrifuged to remove bacteria, and the supernatant put into a fresh tube and stored at 4° C.

Polymerase chain reaction (PCR) was selected as a technique for screening the phage library. Each PCR reaction was done in a 20 μl reaction volume containing 8.84 μl H₂O, 2 μl 10× PCR buffer II (Perkin-Elmer), 2 μl of 25 mM MgCl₂, 0.8 μl 10 mM dNTP mixture (dATP, dCTP, dGTP, dCTP), 0.12 μl of primer LW1129 (approx. 1 μg/μl), 0.12 μl of primer LW1130, 0.12 μl AmpliTaq Gold polymerase (5 U/μl, Perkin Elmer) and 2 μl of phage from each of the 24 tubes. The PCR reaction involved 1 cycle at 95° C. for 10 minutes and 80° C. for 30 minutes, followed by 10 cycles at 95° C. for 30 seconds, 50° C. for 2 minutes, 72° C. for 30 seconds, followed by 30 cycles of at 95° C. for 15 seconds, 50° C. for 30 seconds, 72° C. for 30 seconds, followed by a final “cycle” at 72° C. for 5 minutes.

Following PCR cycling, the contents from each reaction tube was loaded onto a 2% agarose gel and electrophoresed adjacent to known size.standards to screen for PCR products of the expected size length indicative of a clone containing the 251 bp portion of Clone 2939091H1 amplified by the two selected primers. Positive clones were identified in one of the 24 tubes.

From the original tube that had given a PCR product of the correct size, a 5 μl phage aliquot was used to set up a series of dilutions that were plated, incubated and filter lifted in the same manner as the original phage library. The PCR reaction was run as above, and the plate of the lowest dilution to give a PCR product of the expected size was selected for subsequent experiments. This culture tube was again subdivided, filter lifted, and screened using the same PCR procedure. The series of dilutions and subdividing of the plate was continued until a single plaque was isolated that gave a positive PCR band.

Once a single plaque was isolated, 20 μl phage supernatant was used to infect 250 μl of 10×K802 cells (resuspended in SM). The cells were cultured at 37° C. for 16. hours, and genomic phage DNA was isolated using Qiagen's Lambda Midi Kit. Sequencing of this genomic DNA identified a methionine-initiated 945 nucleotide open reading frame that included sequence corresponding to Clone 2939091H1. Analysis of the sequence revealed seven hydrophobic regions that were identified as putative transmembrane regions of a novel GPCR designated CON167. The DNA and deduced amino acid sequences of CON167 are set forth in SEQ ID NOs: 1 and 2.

C. Subcloning of the Coding Region of CON167 via PCR

Additional experiments were conducted to subclone the coding region of the CON167 clone into a useful vector. Two additional PCR primers were designed based on the coding region of CON167. The first, Primer LW1168, from 5′ to 3′ (SEQ ID NO: 5):
GCACTAGTAATACGACTCACTATAGGGAGACCACCATGGGAAGATGGGTGAACCAGTCC, includes the 5′ end of the CON167 coding sequence (underlined) as well as upstream sequence of the T7 promoter, useful for subsequent expression work. The second, Primer LW1169, from 5′ to 3′ (SEQ ID NO: 6):
GACTGGATCCCCCGGGCTTTTTTTTTTTTTTTGCGGCCGCTCAGTGCTGGCTGCCAATCC, includes sequence complementary to the 3′ end of the CON167 coding sequence (underlined), followed by sequence useful for subsequent cloning and expression work.

The PCR was performed in a 50 μl reaction containing 35 μl H₂O, 5 μl 10× TT buffer (140 mM Ammonium Sulfate, 0.1% gelatin, 0.6 M Tris-tricine pH 8.4), 5 μl 15 mM MgSO₄, 2 μl 10 mM dNTP, 2 μl genomic phage DNA (0.175 μg/μl), 0.3 μg/μl Primer LW1168 (1 μg/μl), 0.3 μl Primer LW1169 (1 μg/μl), 0.5 μl High Fidelity Taq polymerase (Boehringer Mannheim). The PCR reaction was started with 1 cycle of 94° C. for 2 minutes; followed by 10 cycles at 94° C. for 30 seconds, 55° C. for 2 minutes, and 72° C. for 2 minutes; followed by 25 cycles at 94° C. for 30 seconds, 55° C. for 30 seconds, and 72° C. for 2 minutes.

The contents from the PCR reaction was loaded onto a 1.2% agarose gel and electroeluted. The DNA band of expected size was excised from the gel, placed in GenElute Agarose spin column (Supelco) and spun for 10 minutes at maximum speed. The eluted DNA was ethanol-precipitated and resuspended in 6 μl H₂O for ligation.

The PCR fragment containing the CON167 coding sequence was ligated into a commercial vector using Invitrogen's Original TA Cloning Kit. The ligation reaction consisted of 6 μl DNA, 1 μl 10× ligation buffer, 2 μl of plasmid pCR2.1 (25 ng/μl), Invitrogen), 1 μl T4 DNA ligase (Invitrogen) and was incubated overnight at 14° C. The reaction was heated at 65° C. for 10 minutes to inactivate the enzyme, and then on microliter of the ligation reaction was transformed in One Shot cells (Invitrogen) and plated onto ampicillin plates. A single colony containing an insert was used to inoculate 50 ml culture of LB medium. The culture was grown for. 16 hours at 37° C., and centrifuged into a cell pellet. Plasmid DNA was purified from the pellet using a Qiagen Plasmid Midi Kit and then sequenced to confirm successful cloning of the CON167 insert, using an AB1377 fluorescence-based sequencer (Perkin Elmer/Applied Biosystems Division, PE/ABD, Foster City, Calif.) and the ABI PRISMTM Ready Dye-Deoxy Terminator kit with Taq FSTM polymerase.

Each ABI cycle sequencing reaction contained about 0.5 μg of plasmid DNA. Cycle-sequencing was performed using an initial denaturation at 98° C. for 1 minute, followed by 50 cycles: 98° C. denaturation for 30 seconds, annealing at 50° C. for 30 seconds, and extension at 60° C. for 4 minutes. Temperature cycles and times were controlled by a Perkin-Elmer 9600 thermocycler. Extension products were purified using Centriflex™ gel filtration cartridges (Advanced Genetic Technologies Corp., Gaithersburg, Md.). Each reaction product was loaded by pipette onto the column, which was then centrifuged in a swinging bucket centrifuge (Sorvall model RT6000B table top centrifuge) at 1500×g for 4 minutes at room temperature. Column-purified samples were dried under a vacuum for about 40 minutes and then dissolved in 5 μl of a DNA loading solution (83% deionized formamide, 8.3 mM EDTA, and 1.6 mg/ml Blue Dextran). The samples were then heated to 90° C. for three minutes and loaded into the gel sample wells for sequence analysis by the AB1377 sequencer. Sequencer analysis was done by importing AB1377 files into the Sequencer program (Gene Codes, Ann Arbor, Mich.). Generally sequence reads of 700 bp were obtained. Potential sequence errors were minimized by obtaining sequence information from both DNA strands and by re-sequencing difficult areas using primers at different locations until all sequencing ambiguities were removed.

### EXAMPLE2

### Analysis of the CON167 Sequence

The DNA and deduced amino acid sequence for CON167 are set forth in SEQ ID NO: 1 and 2, respectively. Beginning with the initiator methionine, the CON167 genomic clone contains an open reading frame of 945 nucleotides encoding 315 amino acids, followed by a stop codon. Using a FORTRAN computer program called “tmtrest.all” [Parodi et al., Comput. Appl. Biosci., 5: 527-535 (1994)], CON167 was deduced to contain seven transmembrane-spanning domains corresponding to residues 27-51, 60-79, 92-121, 151-170, 196-220, 242-260, 274-294.

CON167 contains a DHY sequence following the third transmembrane domain (TM3), which distinguishes CON167 from most GPCR which have a DRY sequence at this location. The sequence DHY is not unheard of, however, and has been observed, for example, in the receptor known as GPR1. [See Marchese et al., Genomics, 23:609-618 (1994)].

The sequence of the C0N167 was compared to sequences of known genes. CON167 is 45% identical and 63% similar to a rat olfactory receptor [see Guillaume et al., Recept. Channels, 3: 33-40 (1994)] and 46% identical/63% similar to the human olfactory receptor fat11 [Fan et al., Immunogenetics, 44: 97-103 (1996)]. A subsequent database search revealed that a 223 amino acid stretch of CON167 was 83% identical (187/223) and 87% similar (198/223) to murine olfactory receptor G7 [Krautwurst et al., Cell, 95: 917-926 (1998)]. This level of sequence similarity suggests that CON167 represents an ortholog (species equivalent) of the G7 receptor, but at least two important differences are evident. A Northern hybridization using a CON167 probe showed expression in several areas of the brain including the cerebellum, cerebral cortex and the medulla. Second, following transmembrane III, the CON167 polypeptide has a DHY sequence while the mouse has a DRY. The GPCR known as GPR1 provides a precedence for this observation, where the human gene has a DHY sequence but the rat othologue has a DRY. (See Marchese et al., Biochem. Biophy. Res. Comm., 205: 1952-1958 (1994).) A Northern hybridization showed a different distribution in the brain for the human and rat receptor which the authors believed demonstrated a functional variation for this receptor in these two species.

### EXAMPLE 3

### Hybridization Analysis Demonstrates that CON167 is Expressed in the Brain

Hybridization analyses were performed as follows to determine cells/tissues in which the CON167 gene is expressed. The full-length CON167 coding insert DNA was labeled (³²P, specific activity=1.1×10⁹ cpm/ug) with the Prime-It II labeling kit from Stratagene, following the supplier's protocol. Human multiple tissue northern blots (Clontech 7755-1 and 7760-1, polyA+ RNA) were hybridized with the CON167 probe and washed according to the supplier's protocol (ExpressHyb solution, high stringency (68° C.), and the blots were analyzed on a phosphorimager (Molecular Dynamics). The resulting image revealed a signal at about 2.2 kb in the following tissues: heart, brain, placenta, lung, skeletal-muscle, kidney, and pancreas. Expression appeared to be highest in the heart, placenta, and liver. The human brain blot gave a positive signal for the cerebellum, cerebral cortex, medulla, spinal cord, occiptal pole, frontal lobe, temporal lobe, and the putamen. Expression appeared to be highest in the cerebellum, cerebral cortex, and medulla. Expression of CON167 in the brain provides an indication that modulators of CON167 activity have utility for-treating neurological disorders, including but not limited to schizophrenia, depression, anxiety, bipolar disease, epilepsy, neuritis, neurasthenia, neuropathy, neuroses, and the like. Use of CON167 modulators, including CON167 ligands and anti-CON167 antibodies, to treat individuals having such disease states is intended as an aspect of the invention.

An additional signal of about 2.7 kb was observed in heart and skeletal muscle mRNA. Expression in these muscular tissues provides an indication that modulators of CON167 activity have utility for treating cardiovascular or skeletal muscle disease states, including but not limited to congestive heart failure, restenosis, muscular dystrophy, myositis, myopathies, myasthenias, and the like. Use of CON167 modulators, including ligands and anti-CON167 antibodies, to treat individuals having such disease states is intended as an aspect of the invention.

### EXAMPLE 4

Recombinant Expression of CON167 in Eukaryotic Host Cells

To produce CON167 protein, a CON167-encoding polynucleotide is expressed in a suitable host cell using a suitable expression vector, using standard genetic engineering techniques. For example, the CON167-encoding sequence described in Example 1 is subcloned into the commercial expression vector pzeoSV2 (Invitrogen, San Diego, Calif.) and transfected into Chinese Hamster Ovary (CHO) cells using the transfection reagent fuGENE 6 (Boehringer-Mannheim) and the transfection protocol provided in the product insert. Alternative eukaryotic cell lines, such as African Green Monkey Kidney cells (COS-7, ATCC CRL-1651) or human kidney cells, (e.g., HEK-293, ATCC CRL-1573)may be employed. Cells stably expressing Con 167 are selected by growth in the presence of 100 ug/ml zeocin (Stratagene, LaJolla, Calif.). Optionally, the CON167 is purified from the cells using standard chromatographic techniques. To facilitate purification, antisera is raised against one or more synthetic peptide sequences that correspond to portions of the CON167 amino acid sequence, and the antisera is used to affinity purify CON167. The CON167 also may be expressed in frame with a tag sequence (e.g., polyhistidine, hemaggluttinin, FLAG) to facilitate purification. Moreover, it will be appreciated that many of the uses for CON 167 polypeptides, such as assays described below, do not require purification of CON167 from the host cell.

### EXAMPLE 5

### Assays to Identify Modulators of CON167 Activity

Set forth below are assays for identifying modulators (agonists and antagonists) of CON167 activity. Among the modulators that can be identified by these assays include natural ligand compounds of the receptor; synthetic analogs and derivatives of natural ligands; antibodies, antibody fragments, and/or antibody-like compounds derived from natural antibodies or from antibody-like combinatorial libraries; and/or synthetic compounds identified through high throughput screening of libraries; and the like. All modulators that bind CON167 are useful for identifying CON167 in tissue samples (e.g., for diagnostic purposes, pathological purposes, and the like). Agonist and antagonist modulators are useful for up-regulating and down-regulating CON167 activity, respectively, to treat disease states characterized by abnormal levels of CON167 activity. CON167 binding molecules also may be used to deliver a therapeutic compound or a label to cells that express CON167 (e.g., by attaching the compound or label to the binding molecule). The assays may be performed using single putative modulators, and/or may be performed using a known agonist in combination with candidate antagonists (or visa versa).

A. cAMP Assays

In one type of assay, levels of cyclic adenosine monophosphate (cAMP) are measured in CON167-transfected cells that have been exposed to candidate modulator compounds. Protocols for cAMP assays have been described in the literature. [See, e.g., Sutherland et al., Circulation, 37: 279 (1968); Frandsen, E. K. and Krishna, G, Life Sciences, 18: 529-541 (1976); Dooley et al., Journal of Pharmacology and Experimental Therapeutics, 283 (2): 735-41 (1997); and George et al., Journal of Biomolecular Screening, 2 (4): 235-40 (1997).] An exemplary protocol for such an assay, using an. Adenylyl Cyclase Activation FlashPlate® Assay from NEN™ Life Science Products, is set forth below.

Briefly, the CON167 coding sequence (e.g., a cDNA or intronless genomic DNA) is subcloned into a commercial expression vector, such as pzeoSV2 (Invitrogen, San Diego, Calif.), and transiently transfected into Chinese Hamster Ovary (CHO) cells using known methods, such as the transfection reagent FuGENE 6 (Boehringer-Mannheim) and the transfection protocol provided in the product insert. The transfected CHO cells are seeded into the 96 well microplates from the FlashPlate® assay kit, which are coated with solid scintillant to which antisera to cAMP has been bound. For a control, some wells are seeded with wild type (untransfected) CHO cells. Other wells on the plate receive various amounts of cAMP standard solution for use in creating a standard curve.

One or more test compounds are added to the cells in each well, with water and/or compound-free media/diluent serving as a control. After treatment, cAMP is allowed to accumulate in the cells for exactly 15 minutes at room temperature. The assay is terminated by the addition of lysis buffer containing [¹²⁵I]-labelled cAMP, and the plate is counted using a Packard Topcount™ 96-well microplate scintillation counter. Unlabelled cAMP from the lysed cells (or from standards) competes with the fixed amounts of [¹²⁵I]-cAMP for antibody bound to the plate. A standard curve is constructed, and cAMP values for the unknowns are obtained by interpolation. Changes in intracellular cAMP level of the cells in response to exposure to a test compound are indicative of CON167 modulating activity. Modulators that act as agonists at receptors which couple to the Gs subtype of g-proteins will stimulate production of cAMP, leading to a measurable 3-10 fold increase. Receptor agonists which couple to the Gi/o subtype of g-proteins will inhibit forskolin-stimulated cAMP production, leading to a measurable decrease of 50-100%. Modulators that act as inverse agonists will reverse these effects at receptors that are either constitutively active or activated by known agonists.

B. Aequorin Assays

In another assay cells (e.g., CHO cells) are transiently co-transfected with both a CON167 expression construct and a construct that encodes the photoprotein apoaquorin. In the presence of the cofactor coelenterazine, apoaquorin will emit a measurable luminescence that is proportional to the amount of intracellular (cytoplasmic) free calcium. [See generally Cobbold P. H. and Lee, J. A. C. “Aequorin measurements of cytoplasmic free calcium. In: McCormack J. G. and Cobbold P. H., eds., Cellular Calcium: A Practical Approach. Oxford: IRL Press (1991); Stables et al., Analytical Biochemistry, 252: 115-26 (1997); and Haugland, R. P. Handbook of Fluorescent Probes and Research Chemicals. Sixth edition. Eugene Oreg.: Molecular Probes (1996).]

In one exemplary assay, CON167 is subcloned into the commercial expression vector pzeoSV2 (Invitrogen, San Diego, Calif.) and transiently co-transfected along with a construct that encodes the photoprotein apoaequorin (Molecular Probes, Eugene, Oreg.) into CHO cells using the transfection reagent FuGENE 6 (Boehringer-Mannheim) and the transfection protocol provided in the product insert.

The cells are cultured for 24 hours at 37° C. in alpha MEM (Gibco/BRL, Gaithersburg, Md.) supplemented with 10% fetal bovine serum, 2 mM glutamine, 10 U/ml penicillin and 10 μg/ml streptomycin, at which time the media is changed to serum-free αMEM containing 5 μM coelenterazine (Molecular Probes, Eugene, Oreg.), and the cells are cultured for two additional hours at 37° C. Cells are then detached from the plate using VERSEN (Gibco/BRL), washed and resuspended at 200,000 cells/ml in serum-free αMEM.

Dilutions of candidate CON167 modulator drugs are prepared in serum-free αMEM and dispensed into wells of an opaque 96-well assay plate, 50 μl/well. Plates are loaded onto an MLX microtiter plate luminometer (Dynex Technologies, Inc., Chantilly, Va.). The instrument is programmed to dispense 50 μl cell suspension into each well, one well at a time, and immediately read luminescence for 15 seconds. Dose-response curves for the modulator candidates are constructed using the area under the curve for each light signal peak. Data are analyzed with Slide Write, using the equation for 1-site ligand, and EC₅₀ values are obtained. Changes in luminescence caused by the drugs are considered indicative of modulatory activity. Modulators that act as receptor agonists which couple to the Gq subtype of g-proteins give an increase in luminescence of up to 100 fold. Modulators that act as inverse agonists will reverse this effect at receptors that are either constitutively active or activated by known agonists.

C. Luciferase Reporter Gene Assay

The photoprotein luciferase provides another useful tool for assaying for modulators of CON167 activity. Cells (e.g., CHO cells or COS 7 cells) are transiently co-transfected with both a CON167 expression construct (e.g., CON167 in pzeoSV2 (Invitrogen, San Diego, Calif.)) and a reporter construct which includes a gene for the luciferase protein downstream from a transcription factor, either cAMP-response element (CRE), AP-1, or NF kappa B. Agonist binding to receptors coupled to the Gs subtype of g-proteins leads to increases in cAMP, activating the CRE transcription factor and resulting in expression of the luciferase gene. Agonist binding to receptors coupled to the Gq subtype of g-protein leads to production of diacylglycerol that activates protein kinase C. As a result, the AP-1 or NF kappa B transcription factors are activated which stimulate expression of the luciferase gene. Expression levels of luciferase reflect the activation status of the signaling events. [See generally George et al., Journal of Biomolecular Screening, 2(4): 235-40 (1997); and Stratowa et al., Current Opinion in Biotechnology, 6: 574-81 (1995).] Luciferase activity may be quantitatively measured using, e.g., luciferase assay reagents that are commercially available from Promega (Madison, Wis.).

In one exemplary assay, CHO cells are plated to 24-well culture dishes at a density of 100,000 cells/well one day prior to transfection and cultured at 37° C. in αMEM (Gibco/BRL, Gaithersburg, Md.) supplemented with 10% fetal bovine serum, 2 mM glutamine, 10 U/ml penicillin and 10 μg/ml streptomycin. Cells are transiently co-transfected with both a CON167 expression construct and a reporter construct containing the luciferase gene. The reporter plasmids CRE-luciferase, AP-1-luciferase and NF kappa B-luciferase may be purchased from Stratagene (LaJolla, Calif.). Transfections are performed using FuGENE 6 transfection reagent (Boehringer-Mannheim), and the protocol provided in the product insert. Cells transfected with the reporter construct alone are used as a control. 24 hours after transfection, cells are washed once with phosphate buffered saline (PBS) pre-warmed to 37° C. Serum-free αMEM is then added to the cells either alone (control) or with one or more candidate modulators and the cells are incubated at 37° C. for five hours. Thereafter, cells are washed once with ice cold PBS and lysed by the addition of 100 μl of lysis buffer/well (from luciferase assay kit, Promega, Madison, Wis.). After incubation for 15 minutes at room temperature, 15 μl of the lysate is mixed with 50 μl substrate solution (Promega) in an opaque white 96-well plate, and the luminescence is read immediately on a Wallace model 1450 MicroBeta scintillation and luminescence counter (Wallace Instruments, Gaithersburg, Md.).

Differences in luminescence in the presence versus the absence of a candidate modulator compound are indicative of modulatory activity. Receptors that are either constitutively active or activated by agonists give a 3-20 fold stimulation of luminescence compared to cells transfected with the reporter gene alone. Modulators that act as inverse agonists will reverse this effect.

D. Intracellular Calcium Measurement Using FLIPR

Changes in intracellular calcium levels are another recognized indicator of G protein coupled receptor activity, and such assays can be employed to evaluate modulators of CON167 activity. For example, CHO cells stably transfected with a CON167 expression vector are plated at a density of 4×10⁴ cells/well in Packard black-walled 96-well plates specially designed to isolate fluorescent signal to individual wells. The cells are incubated for 60 minutes at 37° C. in modified Dulbecco's PBS (D-PBS) containing, 36 mg/L of pyruvate and 1 g/L of glucose with the addition of 1% fetal bovine serum and one of four calcium indicator dyes (Fluo-3™ AM, Fluo-4™ AM, Calcium Green™-1 AM, or Oregon Green™ 488 BAPTA-1 AM) at a concentration of 4 μM. Plates are washed once with modified D-PBS without 1% fetal bovine serum and incubated for 10 minutes at 37° C. to remove residual dye from the cellular membrane. In addition, a series of washes with modified D-PBS without 1% fetal bovine serum is performed immediately prior to activation of the calcium response.

Calcium response is initiated by the addition of one or more candidate receptor agonist compounds, calcium ionophore A23187 (10 μM), or ATP (4 μM). Fluorescence is measured by Molecular Device's FLIPR with an argon laser, excitation at 488 nm. [See, e.g., Kuntzweiler et al, Drug Development Research, 44(1): 14-20 (1998).] The F-stop for the detector camera was set at 2.5 and the length of exposure was 0.4 msec. Basal fluorescence of cells was measured for 20 seconds prior to addition of agonist, ATP, or A23187, and was subtracted from the response signal. The calcium signal is measured for approximately 200 seconds, taking readings every two seconds. Calcium ionophore and ATP increase the calcium signal 200% above baseline levels. In general, activated orphan GPCRs increase the calcium signal approximately 10-15% above baseline signal.

E. Mitogenesis Assay

In mitogenesis assays, the ability of candidate modulators to induce or inhibit CON167-mediated cell growth is determined. [See, e.g., Lajiness et al., Journal of Pharmacology and Experimental Therapeutics, 267(3): 1573-81 (1993).]For example, CHO cells stably expressing CON167 are seeded into 96-well plates at a density of 5000 cells/well and grown at 37° C. in αMEM supplemented with 10% fetal calf serum for 48 hrs, at which time the cells are rinsed twice with serum-free αMEM. After rinsing, 80 μl of fresh αMEM, or αMEM containing a known mitogen, is added along with 20 μl αMEM containing varying concentrations of one or more test compounds diluted in serum free media. As controls, some wells on each plate receive serum-free media alone, and some receive media containing 10% fetal bovine serum. Untransfected cells or cells transfected with vector alone also may serve as controls.

After culture for 16-18 hours, 1 μCi/well of [³H]-thymidine (cpm) is added to the wells and cells are incubated for an additional 2 hours at 37° C. The cells are trypsinized and harvested onto filter mats with a cell harvester (Tomtec) and the filters are counted in a Betaplate counter. The incorporation of 3H-thymidine in serum-free test wells is compared to the results achieved in cells stimulated with serum. Use of multiple concentrations of test compounds permits creation and analysis of dose-response curves using the non-linear, least squares fit equation: A=B×[C/(D+C)]+G where A is the percent of serum stimulation; B is the maximal effect minus baseline; C is the EC₅₀; D is the concentration of the compound; and G is the maximal effect. Parameters B, C and G are determined by Simplex optimization.

Agonists that bind to the receptor are expected to increase [³H]-thymidine incorporation into cells, showing up to 80% of the response to serum. Antagonists that bind to the receptor will inhibit the stimulation seen with a known agonist by up to 100%.

F. [⁻³⁵S]GTPγS Binding Assay

Because G protein coupled receptors signal through intracellular “G proteins” whose activity involves GTP/GDP binding and hydrolysis, measurement of binding of the non-hydrolyzable GTP analog [⁻³⁵S]GTPγS in the presence and absence of putative modulators provides another indicator of modulator activity. [See, e.g., Kowal, et al., Neuropharmacology, 37: 179-87 (1998).]

In one exemplary assay, cells stably transfected with a CON167 expression vector are grown in 10 cm dishes to subconfluence, rinsed once with 5 ml of ice cold Ca²⁺/Mg²⁺ free PBS, and scraped into 5 ml of the same buffer. Cells are pelleted by centrifugation (500×g, 5 minutes), resuspended in 25 mM Tris, 5 mM EDTA, 5 mM EGTA, pH 7.5 (TEE), and frozen in liquid nitrogen. After thawing, the cells are homogenized using a dounce (one ml TEE per plate of cells), and centrifuged at 1,000×g for 5 minutes to remove nuclei and unbroken cells.

The homogenate supernatant is centrifuged at 20,000×g for 20 minutes to isolate the membrane fraction, and the membrane pellet is washed once with TEE and resuspended in binding buffer (20 mM HEPES, pH 7.5, 150 mM NaCl, 10 mM MgCl₂, 1 mM EDTA). The resuspended membranes can be frozen in liquid nitrogen and stored at −70° C. until use.

Aliquots of cell membranes prepared as described above and stored at −70° C. are thawed, homogenized, and diluted to a concentration of 10-50 μg/ml in buffer containing 20 mM HEPES, 10 mM MgCl₂, 1 mM EDTA, 120 MM NaCl, 10 μM GDP, and 0.2 mM ascorbate. In a final volume of 90 μl, homogenates are incubated with varying concentrations of putative modulator compounds or 100 μM GTP for 30 minutes at 30° C. and then placed on ice. To each sample, 10 μl guanosine 5′-O-(3[³⁵S]thio) triphosphate (NEN, 1200 Ci/mmol), ([³⁵S]-GTPγS), was added to a final concentration of 100-200 pM. Samples are incubated at 30° C. for an additional 30 minutes, and then the reaction is then stopped by the addition of 1 ml of 10 mM HEPES, and 10 mM MgCl₂; (pH 7.4), at 4° C., and filtration.

Samples are filtered over Whatman GF/B filters and filters are washed with 20 ml ice-cold 10 mM HEPES, (pH 7.4) and 10 mM MgCl₂. Filters are counted by liquid scintillation spectroscopy. Nonspecific binding of [³⁵S]-GTPγS is measured in the presence of 100 μM GTP and subtracted from the total. Compounds are selected that modulate the amount of [³⁵S]-GTPγS binding in the cells, compared to untransfected control cells. Activation of receptors by agonists gives up to a five-fold increase in [³⁵S]GTPγS binding. This response is blocked by antagonists.

G. MAP Kinase Activity Assay

Evaluation of MAP Kinase activity in cells expressing a GPCR provide another assay to identify modulators of GPCR activity. [See, e.g., Lajiness et al., Journal of Pharmacology and Experimental Therapeutics, 267(3): 1573-81 (1993); and Boulton et al., Cell, 65: 663-75 (1991).]

In one embodiment, CHO cells stably transfected with CON167 are seeded into 6 well plates at a density of 70,000 cells/well 48 hours prior to the assay. During this time, the cells are cultured at 37° C. in αMEM media supplemented with 10% fetal bovine serum, 2 mM glutamine, 10 U/ml penicillin and 10 μg/ml streptomycin. The cells are serum starved for 1-2 hours prior to the addition of stimulants.

For the assay, the cells are treated with media alone or media containing a putative agonist or phorbal ester-myistoyl acetate (PMA) as a positive control. After treatment, the cells are incubated at 37° C. for varying times. To stop the reaction, the plates are placed on ice, the media is aspirated, and the cells are rinsed with 1 ml of ice-cold PBS containing 1 mM EDTA. Thereafter, 200 μl cell lysis buffer (12.5 mM MOPS, (pH 7.3), 12.5. mM β-glycerophosphate, 7.5 mM MgCl₂, 0.5 mM EGTA, 0.5 mM sodium vanadate, 1 mM benzamidine, 1 mM dithiothreitol, 10 μg/ml leupeptin, 10 μg/ml aprotinin, 2 μg/ml pepstatin A, and 1 μM okadaic acid) is added to the cells. The cells are scraped from the plates and homogenized by 10 passages through a 23¾ gauge needle. The cytosol fraction is prepared by centrifugation at 53,000 rpm for 15 minutes.

Aliquots (5-10 μl containing 1-5 μg protein) of cytosols are mixed with 1 mM MAPK Substrate Peptide (APRTPGGRR (SEQ ID NO: 7), Upstate Biotechnology, Inc., N.Y.) and 50 μM [γ-³²P]ATP, (NEN, 3000 Ci/mmol) diluted to a final specific activity of ˜2000 cpm/pmol in a total volume of 25 μl. The samples are incubated for 5 minutes at 30° C., and reactions are stopped by spotting 20 μl on 2 cm² of Whatman P81 phosphocellulose paper. The filter squares are washed in 4 changes of 1% H₃PO₄, and the squares are counted by liquid scintillation spectroscopy. Equivalent cytosolic extracts are incubated without MAPK substrate peptide, and the cpm from these samples are subtracted from the matched samples with the substrate peptide. The cytosolic extract from each well is used as a separate point. Protein concentrations are determined by a dye binding protein assay (Bio-Rad). Agonist activation of the receptor is expected to result in up to a five fold increase in MAPK enzyme activity. This increase is blocked by antagonists.

H. [³H]Arachidonic Acid Release

The activation of GPCR's also has been observed to potentiate arachidonic acid release in cells, providing yet another useful assay for modulators of GPCR activity. [See, e.g., Kanterman et al., Molecular Pharmacology, 39: 364-9 (1991).] For example, CHO cells that are stably transfected with a CON167 expression vector are plated-in 24-well plates at a density of 15,000 cells/well and grown in αMEM media supplemented with 10% fetal bovine serum, 2 mM glutamine, 10 U/ml penicillin and 10 μg/ml streptomycin for 48 hours at 37° C. before use. Cells of each well are labeled by incubation with [³H]arachidonic acid (Amersham Corp., 210 Ci/mmol) at 0.5 μCi/ml in 1 ml αMEM supplemented with 10 mM HEPES (pH 7.5), and 0.5% fatty-acid-free bovine serum albumin for 2 hours at 37° C. The cells are then washed twice with 1 ml of the same buffer.

Candidate modulator compounds are added in 1 ml of the same buffer, either alone or containing 10 μM ATP (Adenosine 5′-triphosphate) and the cells are incubated at 37° C. for 30 minutes. Buffer alone and mock transfected cells are used as controls. Samples (0.5 ml) from each well are counted by liquid scintillation spectroscopy. Agonists which activate the receptor will lead to potentiation of the ATP-stimulated release of [³H]-arachidonic Acid. This potentiation is-blocked by antagonists.

I. Extracellular Acidification Rate

In yet another assay, the effects of putative modulators of CON167 activity are assayed by monitoring extracellular changes in pH induced by the putative modulators. [See, e.g., Dunlop et al., Journal of Pharmacological and Toxicological Methods, 40(1): 47-55 (1998).]

CHO cells transfected with a CON167 expression vector are seeded into 12-mm capsule cups (Molecular Devices Corp.) at 4×10⁵ cells/cup in αMEM supplemented with 10% fetal bovine serum, 2 mM 1-glutamine, 10 units/ml penicillin, and 10 μg/ml streptomycin. The cells are incubated in this media at 37° C. in 5% CO₂ for 24 hours.

Extracellular acidification rates are measured using a Cytosensor microphysiometer (Molecular Devices Corp.). The capsule cups are loaded into the sensor chambers of the microphysiometer and the chambers are perfused with running buffer (bicarbonate free αMEM supplemented with 4 mM 1-glutamine, 10 units/ml penicillin, 10 μg/ml streptomycin, 26 mM NaCl) at a flow rate of 100 μl/min. Agonists or other agents are diluted into the running buffer and perfused through a second fluid path. During each 60 second pump cycle, the pump is run for 38 seconds and is off for the remaining 22 seconds. The pH of the running buffer in the sensor chamber is recorded during the cycle from 43-58 seconds and the pump is re-started at 60 seconds to start the next cycle. The rate of acidification of the running buffer during the recording time is calculated by the Cytosoft program. Changes in the rates of acidification are calculated by subtracting the baseline value (the average of 4 rate measurements immediately before addition of modulator candidates) from the highest rate measurement obtained after addition of a modulator candidate. The selected instrument detects 61 mV/pH unit. Modulators that act as agonists at the receptor result in an increase in the rate of extracellular acidification as compared to the rate in the absence of agonist. This response is blocked by modulators which act as antagonists at the receptor.

### EXAMPLE 6

### Hybridization Analysis Demonstrates that CON167 is Expressed in the Brain

In situ hybridization experiments were performed to analyze the expression pattern of CON167 in the brain. Coronal and sagittal oriented rat brain sections were cryosectioned (20 μm thick) using a Leica CM3050 cryostat. The individual sections were thaw-mounted onto silanated, nuclease-free slides (CEL Associates, Inc., Houston, Tex.), and stored at −80° C. The sections were processed starting with post-fixation in cold 4% paraformaldehyde (pH 7.4), rinsed in cold phosphate-buffered saline (PBS), acetylated using acetic anhydride in triethanolamine buffer, and dehydrated through 70%, 95%, and 100% alcohol at room temperature (RT). Following dehydration, the sections were subjected to delipidation in chloroform, and then rehydration in 100% and 95% alcohol at RT. Sections were allowed to air dry prior to hybridization.

Two antisense oligonucleotides were designed based the cDNA sequence of CON167 (SEQ ID NO: 1) plus the 5′ untranslated region (SEQ ID NO: 10). These oligonucleotides were obtained from Sigma-Genosys (St. Louis, Mo.) and used as probes for in situ hybridization. The first oligonucleotide, designated CON167-1031 has the sequence 5′TGAGGATGGGATAGTGAAGTGGGTGGCTAACGGCCACGTAGTG3′ (SEQ ID NO: 11) which corresponds to the complement of nucleotides 367-409 of SEQ ID NO: 1. The second oligonucleotide, designated CON167-570, has the sequence 5′TGACATGTCTCTATTGTGCTCCAAATTCTTCAGTTCAACAGCGTATGCTC3′ (SEQ ID NO: 12) which corresponds to the complement of nucleotides −102 to 53 of SEQ ID NO: 10. Both oligonucleotides, CON167-1031 and CON167-570, were reconstituted with 1× TE buffer to a concentration of 20 pMol/μl and labeled with ³³P-dATP to yield a specific activity of 3.05×10⁶ and 2.49×10⁶ cpm/μl, respectively.

For use in the hybridization experiments, both oligoprobes were denatured at 70° C. for 3 minutes and pooled in an aqueous hybridization buffer which contained 50% formamide, 10% dextran, 0.3 M NaCl, 10 mM Tris (pH 8.0), 1 mM EDTA, 1× Denhardt's, and 200 mM DTT. The final concentration of both probes in the hybridization buffer was 2 pmol/ml. Sequential brain cryosections were hybridized with 45 μl/slide of the sense and antisense oligoprobe hybridization mixture and then covered with silanized, nuclease-free glass coverslips. The sections were hybridized overnight (15-18 hours) at 37° C. in an incubator.

Following the hybridizations, the coverslips were washed off the slides with 1× SSC for 45 minutes. The slides were then washed for 20 minutes at RT in 1× SSC followed by three high stringency washes in 1× SSC at 65° C. for 20 minutes. Subsequently, the slides washed two additional times in 1× SSC for 45 minutes at RT. After washing, the slides were dehydrated with 70% and 95% ethanol containing 0.3 mM NH₄OAc, and then 100% ethanol, air-dried, and exposed to Kodak BioMax MR-1 film. After 15 days of exposure, the film was developed. Based on these results, sections that showed a hybridization signal on film autoradiography were coated with Kodak NTB-2 nuclear track emulsion and stored in the dark for 30 days. The slides were then developed with Kodak D-19 developer and fixer, then counterstained with hematoxylin 2 (Richard-Allen Scientific, Kalamazoo, Mich.). Emulsion-coated sections were analyzed microscopically to determine the specificity of labeling. The signal was judged to be specific if autoradiographic grains (generated by antisense probe hybridization) were associated clearly with crystal violet stained cell bodies. Autoradiographic grains found between cell bodies were deemed non-specific.

Specific labeling with the antisense probe showed wide spread distribution of CON167 mRNA in the rat brain. Labeled regions included the piriform cortex, hippocampus, and hypothalamus (SON-supra optic nucleus, SCN-supra chiasmatic nucleus). The sense probe did not generate specific labeling.

The observed regional distribution of CON167 mRNA provides a therapeutic indication for natural ligands for CON167 as well as modulators of CON167 activity, such as anti-CON167 antibody substances or small molecules that mimic, agonize or antagonize ligand-mediated CON167 signaling. In particular, the expression pattern provides an indication that such molecules will have utility for treating neurological and/or psychiatric diseases, including but not limited to schizophrenia, affective disorders, attention deficit hyperactivity disorder/attention deficit disorder, depression, anxiety, bipolar disease, epilepsy, neuritis, neurasthenia, neuropathy, neuroses, Alzheimer's disease, Parkinson's Disease, migraine, senile dementia, and the like. Use of CON167 modulators, including CON167 ligands and anti-CON167 antibodies, to treat individuals having such disease states is intended as an aspect of the invention. Such modulators are administered by any means effective to safely deliver the modulators to the CON167-expressing cells, including but not limited to oral administration, inhalation, or injection of compositions comprising the modulators in a pharmaceutically acceptable diluent, adjuvant, or carrier. Efficacy of treatment can initially be determined in any accepted animal model that provides a biochemical or behavioral marker that correlates with disease severity or treatment efficacy.

### Example 6

### Antibodies to CON167

Standard techniques are employed to generate polyclonal or monoclonal antibodies to the CON167 receptor, and to generate useful antigen-binding fragments thereof or variants thereof, including “humanized” variants. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988); and other documents cited below. In one embodiment, recombinant CON167 polypeptides (or cells or cell membranes containing such polypeptides) are used as an antigen to generate the antibodies. In another embodiment, one or more peptides having amino acid sequences corresponding to an immunogenic portion of CON167 (e.g., 6, 7, 8, 9, 10, 11, 12, 13, 14, 15, 16, 17, 18, 19, 20, or more amino acids) are used as antigen. Peptides corresponding to extracellular portions of CON167, especially hydrophilic extracellular portions, are preferred. The antigen may be mixed with an adjuvant or linked to a hapten to increase antibody production.

A. Polyclonal or Monoclonal Antibodies

As one exemplary protocol, recombinant CON167 or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanine; (Pierce), according to the manufacturer's recommendations. For an initial injection, the antigen is emulsified with Freund's Complete Adjuvant and injected subcutaneously. At intervals of two to three weeks, additional aliquots of CON167 antigen are emulsified with Freund's Incomplete Adjuvant and injected subcutaneously. Prior to the final booster injection, a serum sample is taken from the immunized mice and assayed by Western blot to confirm the presence of antibodies that immunoreact with CON167. Serum from the immunized animals may be used as a polyclonal antisera or used to isolate polyclonal antibodies that recognize CON167. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75mM Hepes, pH 8.0) (Boehringer Mannheim) is stirred into the pellet, followed by the addition of serum-free RPMI. Thereafter, the cells are centrifuged and resuspended in RPMI containing 15% FBS, 100 μM sodium hypoxanthine, 0.4 μM aminopterin, 16 μM thymidine (HAT) (Gibco), 25 units/ml of IL-6 (Boehringer Mannheim) and 1.5×10⁶ thymocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusions are screened by ELISA, testing for the presence of mouse IgG that binds to CON167. Selected fusion wells are further cloned by dilution until monoclonal cultures producing anti-CON167 antibodies are obtained.

B. Humanization of Anti-CON167 Monoclonal Antibodies

The expression patterns of CON167 as reported herein and the proven track record of GPCR's as targets for therapeutic intervention suggest therapeutic indications for CON167 inhibitors (antagonists). CON167-neutralizing antibodies comprise one class of therapeutics useful as antagonists. Following are protocols to improve the utility of anti-CON167 monoclonal antibodies as therapeutics in humans, by “humanizing” the monoclonal antibodies to improve their serum half-life and render them less immunogenic in human hosts (i.e., to prevent human antibody response to non-human anti-CON167 antibodies).

The principles of humanization have been described in the literature and are facilitated by the modular arrangement of antibody proteins. To minimize the possibility of binding complement, a humanized antibody of the IgG4 isotype is preferred.

For example, a level of humanization is achieved by generating chimeric antibodies comprising the variable domains of non-human antibody proteins of interest with the constant domains of human antibody molecules. (See, e.g., Morrison and Oi, Adv. Immunol., 44:65-92 (1989). The variable domains-of CON167 neutralizing anti-CON167 antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater level of humanization, only those portions of the variable region gene fragments that encode antigen-binding complementarity determining regions (“CDR”) of the non-human monoclonal antibody genes are cloned into human antibody sequences. [See, e.g., Jones et al., Nature, 321:522-525 (1986); Riechmann et al., Nature, 332:323-327 (1988); Verhoeyen et al., Science, 239:1534-36 (1988); and Tempest et al., Bio/Technology, 9:266-71 (1991). If necessary, the β-sheet framework of the human antibody surrounding the CDR3 regions also is modified to more closely mirror the three dimensional structure of the antigen-binding domain of the original monoclonal antibody. (See Kettleborough et al., Protein Engin., 4:773-783 (1991); and Foote et al., J. Mol. Biol., 224:487-499 (1992).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Molecular Immunol., 28(4/5):489-98 (1991).

The foregoing approaches are employed using CON167-neutralizing anti-CON167 monoclonal antibodies and the hybridomas that produce them to generate humanized CON167-neutralizing antibodies useful as therapeutics to treat or palliate conditions wherein CON167 expression or ligand-mediated CON167 signaling is detrimental.

C. Human CON167-Neutralizing Antibodies from Phage Display

Human CON167-neutralizing antibodies are generated by phage display techniques such as those described in Aujame et al, Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for CON167-specific phage-antibodies using labelled or immobilized CON167 as antigen-probe.

D. Human CON167-Neutralizing Antibodies from Transgenic Mice

Human CON167-neutralizing antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with a CON167 composition using conventional immunization protocols. Hybridomas are generated using B cells from the immunized mice using conventional protocols and screened to identify hybridomas secreting anti-CON167 human antibodies (e.g., as described above).

While the present invention has been described in terms of specific embodiments, it is understood that variations and modifications will occur to those in the art, all of which are intended as aspects of the present invention. Accordingly, only such limitations as appear in the claims should be placed on the invention.

1. A purified and isolated CON167 seven transmembrane receptor polypeptide comprising the amino acid sequence set forth in SEQ ID NO: 2, or a fragment thereof comprising an epitope specific to said seven transmembrane receptor.

2-4. (canceled)

5. A purified and isolated polypeptide fragment of claim 1 comprising a CON167 seven transmembrane receptor fragment selected from the group consisting of the N-terminal extracellular domain of CON167, transmembrane domains of CON167, extracellular loops connecting transmembrane domains of CON167, intracellular loops connecting transmembrane domains of CON167, the C-terminal cytoplasmic domain of CON167, and fusions thereof.

6 A purified and isolated polynucleotide comprising a nucleotide sequence that encodes a mammalian seven transmembrane receptor, wherein said polynucleotide hybridizes to the nucleotide sequence set forth in SEQ ID NO: 1 or the non-coding strand complementary thereto, under the following hybridization conditions:
(a) hybridization for 16 hours at 42° C. in a hybridization solution comprising 50% formamide, 1% SDS, 1M NaCl, 10% Dextran sulfate and
(b) washing 2 times for 30 minutes at 60° C. in a wash solution comprising 0.1×SSC and 1% SDS.

7. (canceled)

8. A purified and isolated polynucleotide of claim 1 comprising a nucleotide sequence encoding a polypeptide having the amino acid sequence of SEO ID NO: 2.

9-10. (canceled)

11. A vector comprising a polynucleotide according to claim 6.

12-13. (canceled)

14. A host cell stably transformed or transfected with a polynucleotide comprising a nucleotide sequence that encodes the polypeptide or fragment of claim 1 in a manner allowing the expression in said host cell of the CON167 seven transmembrane receptor polypeptide or fragment thereof encoded by the polynucleotide.

15. (canceled)

16. A host cell stably transformed or transfected with a vector according to claim 11 in a manner allowing the expression in said host cell of the CON167 seven transmembrane receptor polypeptide or fragment thereof encoded by the polynucleotide.

17. A method for producing a seven transmembrane receptor polypeptide or fragment thereof comprising a step of growing a host cell according to claims 14, in a nutrient medium under conditions in which the cell expresses the seven transmembrane polypeptide encoded by the polynucleotide.

18. (canceled)

19. An antibody specific for the CON167 seven transmembrane receptor of claim 2.

20-24. (canceled)

25. A cell-free composition comprising polyclonal antibodies, wherein at least one of said antibodies is an antibody according to claim 19.

26. (canceled)

27. A polypeptide comprising a fragment of an antibody according to claim 19, wherein said fragment and said polypeptide bind to the CON167 seven transmembrane receptor.

28. (canceled)

29. A composition comprising a polypeptide according to claim 1 in a pharmaceutically acceptable carrier.

30. A composition comprising a polypeptide according to claim 27 in a pharmaceutically acceptable carrier.

31. A method for modulating activity of a CON167 seven transmembrane receptor according to claim 1 comprising the step of contacting said seven transmembrane receptor with an antibody specific for said seven transmembrane receptor, under conditions wherein the antibody binds the receptor.

32. A method for modulating activity of a CON167 seven transmembrane receptor comprising the step of contacting said seven transmembrane receptor with a polypeptide according to claim 27.

33. A method for treating a neurological disorder comprising the step of administering to a mammal in need of such treatment an amount of a polypeptide according to claim 27 sufficient to modulate ligand binding of CON167 seven transmembrane receptor in neurons of said mammal.

34. A method according to claim 31 wherein said contacting step comprises administering a composition to a mammal, said mammal comprising cells that express the CON167 seven transmembrane receptor, said composition comprising said antibody.

35-36. (canceled)

37. A method according to claim 32 wherein said contacting step comprises administering a composition to a mammal, said mammal comprising cells that express the CON167 seven transmembrane receptor, said composition comprising said polypeptide.

38-39. (canceled)

40. An assay to identify compounds that bind CON167 seven transmembrane receptor, comprising the steps of:
(a) contacting a composition comprising CON167 seven transmembrane receptor with a compound suspected of binding CON167;
(b) measuring binding between the compound and CON167.

41-43. (canceled)

44. A compound identified by the method according to claim 40 that binds CON167 seven transmembrane receptor, wherein the compound is not an antibody or a polypeptide comprising an antigen-binding fragment or an antibody that binds CON167.

45. A method for modulating activity of a CON167 seven transmembrane receptor comprising the step of contacting said seven transmembrane receptor with a compound according to claim 44, under conditions wherein said compound binds to the receptor.

46-48. (canceled)

49. A method for identifying a modulator of binding between a CON167 seven transmembrane receptor and a CON167 binding partner, comprising the steps of:
(a) contacting a CON167 binding partner and a composition comprising a polypeptide of claim 1 or a fragment thereof in the presence and in the absence of a putative modulator compound;
(b) detecting binding between the binding partner and the polypeptide; and
(c) identifying a putative modulator compound in view of decreased or increased binding between the binding partner and the polypeptide in the presence of the putative modulator, as compared to binding in the absence of the putative modulator.

50. A modulator identified by the method according to claim 49 that decreases or increases binding between the binding partner and the CON167 seven transmembrane receptor.

51. A method for modulating activity of a CON167 seven transmembrane receptor comprising the step of contacting said seven transmembrane receptor with a modulator according to claim 50, under conditions wherein the modulator increases or deceases binding between the binding partner and the receptor.

52-54. (canceled)
